(19) United States
(12) Patent Application Publication
(10) Pub. No.: US 2006/0052291 A1
(43) Pub. Date: Mar. 9, 2006

(54) Chemically-modified progenipoietin conjugates

(76) Inventors: Ned R. Siegal, Belleville, IL (US); Rory F. Finn, Manchester, MO (US); Robert L. Hills, St. Charles, MO (US)

Correspondence Address: CAROL M. NIELSEN;WINSTEAD SECHREST & MINICK, P.C., 2400 BANK ONE CENTER, 910 TRAVIS STREET, HOUSTON, TX, 77002, US

(21) Appl. No.: 10/481,935

(22) PCT Filed: Jun. 14, 2002
(86) PCT No.: PCT/US02/18810

§ 371 Date: Dec. 22, 2003

(30) Foreign Application Priority Data
Jun. 22, 2001 (US) 60300362

(51) Int. Cl. A61K 38/18; C07K 14/475; C12P 21/06; C12N 5/08

(52) U.S. Cl. 514/12; 525/54.1; 530/350; 435/372; 435/69.1; 435/320.1

(57) ABSTRACT

The present invention provides a chemically modified Progenipoietins (ProGPs) prepared by binding a water soluble polymer to the protein. The chemically-modified protein according to the present invention may have a much longer lasting neutrophil-increasing activity than that of the un-modified ProGP, enabling reduced dose and scheduling opportunities.

### FIELD OF THE INVENTION

The present invention relates to a chemical modification of Progenipoietins (ProGPs), a family of recombinant proteins, which are multifunctional agonists of flt3 ligand and another hematopoietic growth factor receptor, including but not limited to G-CSF, by which the chemical and/or physiological properties of ProGP can be changed. The PEGylated ProGPs may have a decreased clearance rate, improved stability, decreased antigenicity, or a combination thereof. The family of ProGP proteins is defined as the multifunctional proteins comprising a flt3 receptor agonist and a second hematopoietic growth factor or colony stimulating factor receptor agonist described in WO98/17810, which is incorporated herein in its entirety. The present invention also relates to processes for the modification of ProGP. In addition, the present invention relates to pharmaceutical compositions comprising the modified ProGP. A further embodiment is the use of the modified ProGP to treat hematopoietic disorders.

### BACKGROUND OF THE INVENTION

Progenipoietin may be useful in the treatment of general haematopoietic disorders, including those arising from chemotherapy or from radiation therapy (Mac Vittie, T. J.; et al., Exp. Hematol. (1999), 27(10), 1557-1568). ProGP may also be useful in bone marrow transplantation, wound healing, burn treatment, and the treatment of parasite, bacterial or viral infection.

It is generally observed that physiologically active proteins administered into a body can show their pharmacological activity only for a short period due to their high clearance rate in the body. Furthermore, the relative hydrophobicity of these proteins may limit their stability.

For the purpose of decreasing the clearance rate, improving stability or abolishing antigenicity of therapeutic proteins, some methods have been proposed wherein the proteins are chemically modified with water-soluble polymers. Chemical modification of this type may block effectively a proteolytic enzyme from physical contact with the protein backbone itself, thus preventing degradation. Chemical attachment may effectively reduce renal clearance. Additional advantages include, under certain circumstances, increasing the stability and circulation time of the therapeutic protein, increasing solubility, and decreasing immunogenicity. A review article describing protein modification and fusion proteins is Francis, Focus on Growth Factors 3: 4-10 (May 1992) (published by Mediscript, Mountview Court, Friern Barnet Lane, London N20, OLD, UK).

Poly(alkylene oxide), notably poly(ethylene glycol) (PEG), is one such chemical moiety, which has been used in the preparation of therapeutic protein products (the verb “pegylate” meaning to attach at least one PEG molecule). The attachment of poly(ethylene glycol) has been shown to protect against proteolysis, Sada, et al., J. Fermentation Bioengineering 71: 137-139 (1991), and methods for attachment of certain poly(ethylene glycol) moieties are available. See U.S. Pat. No. 4,179,337, Davis et al., “Non-Immunogenic Polypeptides,” issued Dec. 18, 1979; and U.S. Pat. No. 4,002,531, Royer, “Modifying enzymes with Polyethylene Glycol and Product Produced Thereby,” issued Jan. 11, 1977. For a review, see Abuchowski et al., in Enzymes as Drugs. (J. S. Holcerberg and J. Roberts, eds. pp. 367-383 (1981)).

Other water-soluble polymers have been used, such as copolymers of ethylene glycol/propylene glycol, carboxymethylcellulose, dextran, poly(vinyl alcohol), poly(vinyl pyrrolidone), poly(-1,3-dioxolane), poly(-1,3,6-trioxane), ethylene/maleic anhydride copolymer, and poly-amino acids (either homopolymers or random copolymers).

A number of examples of pegylated therapeutic proteins have been described. ADAGEN®, a pegylated formulation of adenosine deaminase, is approved for treating severe combined immunodeficiency disease. ONCASPAR®, a pegylated L-asparaginase has been approved for treating hypersensitive ALL patients. Pegylated superoxide dismutase has been in clinical trials for treating head injury. Pegylated α-interferon (U.S. Pat. Nos. 5,738,846, 5,382,657) has been tested in phase III clinical trials for treating hepatitis with PEG-Intron (pegitron alfa-2b) approved for the treatment of chronic hepatitis C while another molecule, PEGASYS™, still awaits regulatory approval; pegylated glucocerebrosidase and pegylated hemoglobin are reported to have been in preclinical testing. Another example is pegylated IL-6, EF 0 442 724, entitled, “Modified hIL-6,” which discloses poly(ethylene glycol) molecules added to IL-6.

Another specific therapeutic protein, which has been chemically modified, is granulocyte colony stimulating factor, (G-CSF). G-CSF induces the rapid proliferation and release of neutrophilic granulocytes to the blood stream, and thereby provides therapeutic effect in fighting infection. European patent publication EP 0 401 384, published Dec. 12, 1990, entitled, “Chemically Modified Granulocyte Colony Stimulating Factor,” describes materials and methods for preparing G-CSF to which poly(ethylene glycol) molecules are attached. Modified G-CSF and analogs thereof are also reported in EP 0 473 268, published Mar. 4, 1992, entitled “Continuous Release Pharmaceutical Compositions Comprising a Polypeptide Covalently Conjugated To A Water Soluble Polymer,” stating the use of various G-CSF and derivatives covalently conjugated to a water soluble particle polymer, such as poly(ethylene glycol). A modified polypeptide having human granulocyte colony stimulating factor activity is reported in EP 0 335 423 published Oct. 4, 1989. Provided in U.S. Pat. No. 5,824,784 are methods for N-terminally modifying proteins, including N-terminally chemically modified G-CSF compositions. U.S. Pat. No. 5,824,778 discloses chemically modified G-CSF.

Japanese patent application Hei2 (1990)-30555 discloses chemically modified human IL-3 having decreased antigenicity.

The family of ProGP proteins is disclosed in WO 98/17810.

For poly(ethylene glycol), a variety of means has been used to attach the poly(ethylene glycol) molecules to the protein. Generally, poly(ethylene glycol) molecules are connected to the protein via a reactive group found on the protein.

Amino groups, such as those on lysine residues or at the N-terminus, are convenient for such attachment. For example, Royer (U.S. Pat. No. 4,002,531, above) states that reductive alkylation was used for attachment of poly(ethylene glycol) molecules to an enzyme. EP 0 539 167, published Apr. 28, 1993, Wright, “Peg Imidates and Protein Derivatives Thereof” states that peptides and organic compounds with free amino group(s) are modified with an imidate derivative of PEG or related water-soluble organic polymers. Chamow et al., Bioconjugate Chem. 5: 133-140 (1994) report the modification of CD4 immunoadhesin with monomethoxypoly(ethylene glycol)aldehyde via reductive alkylation. The authors report that 50% of the CD4-Ig was MePEG-modified under conditions allowing control over the extent of pegylation. Ibid. at page 137. The authors also report that the in vitro binding capability of the modified CD4-Ig (to the protein gp 120) decreased at a rate correlated to the extent of MePEGylation Ibid. U.S. Pat. No. 4,904,584, Shaw, issued Feb. 27, 1990, relates to the modification of the number of lysine residues in proteins for the attachment of poly(ethylene glycol) molecules via reactive amine groups.

Many methods of attaching a polymer to a protein involve using a moiety to act as a linking group. However, such moieties may be antigenic. A tresyl chloride method involving no linking group is available, but this method may be difficult to use to produce therapeutic products as the use of tresyl chloride may produce toxic by-products. See Francis et al., In: Stability of protein pharmaceuticals: in vivo pathways of degradation and strategies for protein stabilization (Eds. Ahern. T. and Manning, M. C.) Plenum, New York, 1991) Also, Delgado et al., “Coupling of PEG to Protein By Activation With Tresyl Chloride, Applications In Immunoaffinity Cell Preparation”, in Separations Using Aqueous Phase Systems, Applications In Cell Biology and Biotechnology, Fisher et al., eds. Plenum Press; New York, N.Y., 1989 pp. 211-213.

See also, Rose et al., Bioconjugate Chemistry 2: 154-159 (1991) which reports the selective attachment of the linker group carbohydrazide to the C-terminal carboxyl group of a protein substrate (insulin).

The present invention provides chemically modified ProGP molecules having decreased clearance rate, increased stability, decreased antigenicity, or combinations thereof.

### SUMMARY OF THE INVENTION

The present invention relates to chemically modified ProGPs, which have at least one improved chemical or physiological property selected from but not limited to decreased clearance rate, increased stability, and decreased antigenicity. Thus, as described below in more detail, the present invention has a number of aspects relating to chemically modifying ProGPs as well as specific modifications using a variety of poly(ethylene glycol) moieties.

The present invention also relates to methods of producing the chemically modified ProGPs.

The present invention also relates to compositions comprising the chemically modified ProGPs.

The modified ProGP of the present invention may be useful in the treatment of, but not limited to, neutropenia, thrombocytopenia, mobilization of hematopoietic progenitors and stem cells into peripheral blood, bone marrow suppression or hematopoietic deficiencies, and immunodeficiencies.

### BRIEF DESCRIPTION OF THE DRAWINGS

FIG. 1 is a reproduction of the ion-exchange chromatography elution profile of a 30,000 MW PEG-ALD ProGP-4 reaction.

FIG. 2a is an SEC HPLC profile of recombinant ProGP-4.

FIG. 2b is an SEC HPLC profile of the 30,000 MW PEG-ALD ProGP-4 Reaction mix.

FIG. 2c is an SEC HPLC profile of ion exchange purified N-terminally mono-pegylated 30,000 MW PEG-ALD ProGP-4.

FIG. 3 is an SDS-PAGE of 30000 MW PEG-ALD ProGP-4. Lane 1. MW Protein standards; Lane 2. Blank; Lane 3. ProGP-4; Lane 4. 30,000 MW PEG-ALD ProGP-4.

FIG. 4 is an SDS-SEC HPLC profile of ion exchange purified N-terminally mono-pegylated 30,000 MW PEG-ALD ProGP-4.

FIG. 5 shows a reversed phase HPLC profile for N-terminally mono-PEGylated 30,000 MW PEG-ALD ProGP-4.

FIG. 6 shows a MALDI-TOF spectrum of reversed phase purified monomeric mono-PEGylated 30,000 MW PEG-ALD ProGP-4 monomer.

FIG. 7 shows a SEC HPLC profile of a mono-PEGylated 30,000 MW PEG-ALD ProGP-4 trypsin digest.

FIG. 8 illustrates a comparison of response curves for a flt3 receptor agonist, a G-CSF receptor agonist, co-addition of a flt3 receptor agonist and a G-CSF receptor agonist, un-PEGylated ProGP-4 and mono-PEGylated 30,000 MW PEG-ALD ProGP-4 in a colony forming unit granulocyte/macrophage (CFU-GM) assay which measures expansion and differentiation of a human bone marrow-derived CD34+ cells.

FIG. 9 illustrates a comparison of murine plasma concentration curves for ProGP-4 and mono-PEGylated 30,000 MW PEG-ALD ProGP-4.

FIG. 10 compares the in vivo bioactivity of un-PEGylated ProgP-4 dosed daily in mice to N-terminally mono-PEGylated 30,000 MW PEG-ALD ProGP-4 dosed every three days by illustrating the total white blood cell (WBC) and dendritic cell (DC) counts in peripheral blood and spleen obtained 24 hours following dosing.

FIG. 11 compares the kinetics of the in vivo DC response for un-PEGylated ProgP-4 dosed daily in mice to N-terminally mono-PEGylated 30,000 MW PEG-ALD ProGP-4 dosed every three days by illustrating the total white blood cell(WBC) and dendritic cell (DC) counts in peripheral blood and spleen obtained 24, 48 and 72 hours following dosing.

FIG. 12 shows the amino acid sequence of ProGP-4.

### DETAILED DESCRIPTION OF THE INVENTION

Progenipoietin (ProGP) proteins are members of a family of recombinant proteins, which are multifunctional agonists of flt3 ligand and another hematopoietic growth factor. Their recombinant production and methods of use are detailed in WO 98/17810.

Progenipoietin proteins are of the formula
R₁-L₁-R₂, R₂-L₁-R₁, R₁-R₂, or R₂-R₁

wherein R₁ is a polypeptide comprising; a modified flt-3 ligand amino acid sequence of the Formula:

```
SEQ ID NO:1
ThrGlnAspCysSerPheGlnHisSerProIleSerSerAspPheAlaValLysIleArg
                                                      10 20
GluLeuSerAspTyrLeuLeuGlnAspTyrProValThrValAlaSerAsnLeuGlnAsp
                                                      30 40
GluGluLeuCysGlyGlyLeuTrpArgLeuValLeuAlaGlnArgTrpMetGluArgLeu
                                                      50 60
LysThrValAlaGlySerLysMetGlnGlyLeuLeuGluArgValAsnThrGluIleHis
                                                      70 80
PheValThrLysCysAlaPheGlnProProProSerCysLeuArgPheValGlnThrAsn
                                                      90 100
IleSerArgLeuLeuGlnGluThrSerGluGlnLeuValAlaLeuLysProTrpIleThr
                                                      110 120
ArgGlnAsnPheSerArgCysLeuGluLeuGlnCysGlnProASrSerSerThrLeu
                                                      130
```

wherein the N-terminus is joined to the C-terminus directly or through a linker (L₂) capable of joining the N-terminus to the C-terminus and having new C- and N-termini at amino acids;

28-29
29-30
30-31
31-32
32-33
34-35
36-37
37-38
38-39
39-40
40-41
41-42
42-43
64-65
65-66
66-67
86-87
87-88
88-89
89-90
90-91
91-92
92-93
93-94
94-95
95-96
96-97
97-98
98-99
99-100
100-101
101-102
102-103
respectively;

wherein R₂ is a factor selected from the group consisting of: a colony stimulating factor, a cytokine, a lymphokine, an interleukin, and a hematopoietic growth factor;

wherein L₁ is a linker capable of linking R₁ to R₂.

The Progenipoietin protein can optionally be immediately preceded by (methionine⁻¹), (alanine⁻¹) or (methionine⁻², alanine⁻¹).

In a preferred embodiment the Progenipoietin proteins are of the formula:
R₁-L₁-R₂, R₂-L₁-R₁, R₁-R₂, or R₂-R₁

wherein R₁ is a polypeptide comprising; a modified flt-3 ligand amino acid sequence of the Formula:

```
SEQ ID NO:1
ThrGlnAspCysSerPheGlnHisSerProIleSerSerAspPheAlaValLysIleArg
                                                      10 20
GluLeuSerAspTyrLeuLeuGlnAspTyrProValThrValAlaSerAsnLeuGlnAsp
                                                      30 40
GluGluLeuCysGlyGlyLeuTrpArgLeuValLeuAlaGlnArgTrpMetGluArgLeu
                                                      50 60
LysThrValAlaGlySerLysMetGlnGlyLeuLeuGluArgValAsnThrGluIleHis
                                                      70 80
PheValThrLysCysAlaPheGlnProProProSerCysLeuArgPheValGlnThrAsn
                                                      90 100
IleSerArgLeuLeuGlnGluThrSerGluGlnLeuValAlaLeuLysProTrpIleThr
                                                      110 120
ArgGlnAsnPheSerArgCysLeuGluLeuGlnCysGlnProASrSerSerThrLeu
                                                      130
```

wherein the N-terminus is joined to the C-terminus directly or through a linker (L₂) capable of joining the N-terminus to the C-terminus and having new C- and N-termini at amino acids;

28-29
29-30
30-31
31-32
32-33
34-35
36-37
37-38
38-39
39-40
40-41
41-42
42-43
64-65
65-66
66-67
86-87
87-88
88-89
89-90
90-91
91-92
92-93
93-94
94-95
95-96
96-97
97-98
98-99
99-100
100-101
101-102
102-103
respectively;

wherein R₂ is a polypeptide, comprising; a modified human G-CSF amino acid sequence of the formula:

```
SEQ ID NO:260
1 10
Xaa Xaa Xaa Gly Pro Ala Ser Ser Leu Pro Gln Ser Xaa
                                                20
Leu Leu Xaa Xaa Xaa Glu Gln Val Xaa Lys Xaa Gln Gly Xaa Gly
        30 40
Ala Xaa Leu Gln Glu Xaa Leu Xaa Ala Thr Tyr Lys Leu Xaa Xaa
                                                50
Xaa Glu Xaa Xaa Val Xaa Xaa Gly His Ser Xaa Gly Ile Pro Trp
        60 70
Ala Pro Leu Ser Ser Xaa Pro Ser Xaa Ala Leu Xaa Leu Ala Gly
                                                80
Xaa Leu Ser Gln Leu His Ser Gly Leu Phe Leu Tyr Gln Gly Leu
        90 100
Leu Gln Ala Leu Glu Gly Ile Ser Pro Glu Leu Gly Pro Thr Leu
                                                110
Xaa Thr Leu Gln Xaa Asp Val Ala Asp Phe Ala Xaa Thr Ile Trp
        120 130
Gln Gln Met Glu Xaa Xaa Gly Met Ala Pro Ala Leu Gln Pro Thr
                                                140
Gln Gly Ala Met Pro Ala Phe Ala Ser Ala Xaa Gln Xaa Xaa Ala
        150 160
Gly Gly Val Leu Val Ala Ser Xaa Leu Gln Xaa Phe Leu Xaa Xaa
                                                170
Ser Tyr Arg Val Leu Xaa Xaa Leu Ala Gln Pro
```

wherein
Xaa at position 1 is Thr, Ser, Arg, Tyr or Gly;
Xaa at position 2 is Pro or Leu;
Xaa at position 3 is Leu, Arg, Tyr or Ser;
Xaa at position 13 is Phe, Ser, His, Thr or Pro;
Xaa at position 16 is Lys, Pro, Ser, Thr or His;
Xaa at position 17 is Cys, Ser, Gly, Ala, Ile, Tyr or Arg;
Xaa at position 18 is Leu, Thr, Pro, His, Ile or Cys;
Xaa at position 22 is Arg, Tyr, Ser, Thr or Ala;
Xaa at position 24 is Ile, Pro, Tyr or Leu;
Xaa at position 27 is Asp, or Gly;
Xaa at position 30 is Ala, Ile, Leu or Gly;
Xaa at position 34 is Lys or Ser;
Xaa at position 36 is Cys or Ser;
Xaa at position 42 is Cys or Ser;
Xaa at position 43 is His, Thr, Gly, Val, Lys, Trp, Ala, Arg, Cys, or Leu;
Xaa at position 44 is Pro, Gly, Arg, Asp, Val, Ala, His, Trp, Gln, or Thr;
Xaa at position 46 is Glu, Arg, Phe, Arg, Ile or Ala;
Xaa at position 47 is Leu or Thr;
Xaa at position 49 is Leu, Phe, Arg or Ser;
Xaa at position 50 is Leu, Ile, His, Pro or Tyr;
Xaa at position 54 is Leu or His;
Xaa at position 64 is Cys or Ser;
Xaa at position 67 is Gln, Lys, Leu or Cys;
Xaa at position 70 is Gln, Pro, Leu, Arg or Ser;
Xaa at position 74 is Cys or Ser;
Xaa at position 104 is Asp, Gly or Val;
Xaa at position 108 is Leu, Ala, Val, Arg, Trp, Gln or Gly;
Xaa at position 115 is Thr, His, Leu or Ala;
Xaa at position 120 is Gln, Gly, Arg, Lys or His
Xaa at position 123 is Glu, Arg, Phe or Thr
Xaa at position 144 is Phe, His, Arg, Pro, Leu, Gln or Glu;
Xaa at position 146 is Arg or Gln;
Xaa at position 147 is Arg or Gln;
Xaa at position 156 is His, Gly or Ser;
Xaa at position 159 is Ser, Arg, Thr, Tyr, Val or Gly;
Xaa at position 162 is Glu, Leu, Gly or Trp;
Xaa at position 163 is Val, Gly, Arg or Ala;
Xaa at position 169 is Arg, Ser, Leu, Arg or Cys;
Xaa at position 170 is His, Arg or Ser;
   wherein 1-11 amino acids from the N-terminus and/or 1-5 amino acids from the C-terminus are optionally deleted from said modified human G-CSF amino acid sequence; and

wherein the N-terminus is joined to the C-terminus directly or through a linker (L₂) capable of joining the N-terminus to the C-terminus and having new C- and N-termini at amino acids;

38-39
39-40
40-41
41-42
42-43
43-44
45-46
48-49
49-50
52-53
53-54
54-55
55-56
56-57
57-58
58-59
59-60
60-61
61-62
62-63
63-64
64-65
65-66
66-67
67-68
68-69
69-70
70-71
71-72
91-92
92-93
93-94
94-95
95-96
96-97
97-98
98-99
99-100
123-124
124-125
125-126
126-127
128-129
128-129
129-130
130-131
131-132
132-133
133-134
134-135
135-136
136-137
137-138
138-139
139-140
140-141
141-142
or 142-143
respectively;

wherein L₁ is a linker capable of linking R₁ to R₂.

In another embodiment the progenipoietin can optionally be immediately preceded by (methionine⁻¹), (alanine⁻¹) or (methionine⁻², alanine⁻¹).

In a preferred embodiment R₁ is selected from SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:169, SEQ ID NO:170, SEQ ID NO:171, SEQ ID NO:172, SEQ ID NO:173, SEQ ID NO:174, and SEQ ID NO:175.

In a preferred embodiment R₂ is GM-CSF, G-CSF, G-CSF Ser¹⁷, c-mpl ligand (TPO), M-CSF, erythropoietin (EPO), IL-1, IL-4, IL-2, IL-3, IL-3 variant, IL-5, IL 6, IL-7, IL-8, IL-9, IL-10, IL-11, IL-12, IL-13, IL-15, LIF, flt3/flk2 ligand, human growth hormone, B-cell growth factor, B-cell differentiation factor, eosinophil differentiation factor and stem cell factor (SCF).

In a preferred embodiment the Progenipoietin protein is selected from SEQ ID NO:32, SEQ ID NO:33, SEQ ID NO:34, SEQ ID NO:35, SEQ ID NO:36, SEQ ID NO:37, 38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, SEQ ID NO:57, SEQ ID NO:58, SEQ ID NO:59, SEQ ID NO:60, SEQ ID NO:61, SEQ ID NO:62, SEQ ID NO:63, SEQ ID NO:64, SEQ ID NO:65, SEQ ID NO:66, SEQ ID NO:67, SEQ ID NO:68, SEQ ID NO:69, SEQ ID NO:70, SEQ ID NO:71, SEQ ID NO:72, SEQ ID NO:73, SEQ ID NO:74, SEQ ID NO:75, SEQ ID NO:76, SEQ ID NO:77, SEQ ID NO:78, SEQ ID NO:79, SEQ ID NO:80, SEQ ID NO:81, SEQ ID NO:82, SEQ ID NO:83, SEQ ID NO:84, SEQ ID NO:85, SEQ ID NO:86, SEQ ID NO:87, SEQ ID NO:88, SEQ ID NO:89, SEQ ID NO:90, SEQ ID NO:91, SEQ ID NO:92, SEQ ID NO:93, SEQ ID NO:94, SEQ ID NO:95, SEQ ID NO:96, SEQ ID NO:97, SEQ ID NO:98, SEQ ID NO:99, SEQ ID NO:100, SEQ ID NO:101, SEQ ID NO:102, SEQ ID NO:103, SEQ ID NO:104, SEQ ID NO:105, SEQ ID NO:106, SEQ ID NO:107, SEQ ID NO:108, SEQ ID NO:109, SEQ ID NO:110, SEQ ID NO:111, SEQ ID NO:112, SEQ ID NO:113, SEQ ID NO:114, SEQ ID NO:115, SEQ ID NO:116, SEQ ID NO:117, SEQ ID NO:118, SEQ ID NO:119, SEQ ID NO:120, SEQ ID NO:121, SEQ ID NO:122, SEQ ID NO:123, SEQ ID NO:124, SEQ ID NO:125, SEQ ID NO:126, SEQ ID NO:127, SEQ ID NO:128, SEQ ID NO:129, SEQ ID NO:130, SEQ ID NO:131, SEQ ID NO:132, SEQ ID NO:133, SEQ ID NO:134, SEQ ID NO:135, SEQ ID NO:136, SEQ ID NO:137, SEQ ID NO:138, SEQ ID NO:139, SEQ ID NO:140, SEQ ID NO:141, SEQ ID NO:142, SEQ ID NO:143, SEQ ID NO:144, SEQ ID NO:145, SEQ ID NO:146, SEQ ID NO:147, SEQ ID NO:148, SEQ ID NO:149, SEQ ID NO:150, SEQ ID NO:151, SEQ ID NO:152, SEQ ID NO:153, SEQ ID NO:154, SEQ ID NO:155, SEQ ID NO:1556, SEQ ID NO:157, SEQ ID NO:158, SEQ ID NO:159, SEQ ID NO:160, SEQ ID NO:161, SEQ ID NO:162, SEQ ID NO:163, SEQ ID NO:164, SEQ ID NO:165, SEQ ID NO:166, SEQ ID NO:167, SEQ ID NO:168, SEQ ID NO:176, SEQ ID NO:177, SEQ ID NO:178, SEQ ID NO:179, SEQ ID NO:180, SEQ ID NO:181, SEQ ID NO:182, SEQ ID NO:183, SEQ ID NO:184, SEQ ID NO:185, SEQ ID NO:186, SEQ ID NO:187, SEQ ID NO:188, SEQ ID NO:189, SEQ ID NO:190, SEQ ID NO:191, SEQ ID NO:192, SEQ ID NO:193, SEQ ID NO:194, SEQ ID NO:195, SEQ ID NO:196, SEQ ID NO:197, SEQ ID NO:198, SEQ ID NO:199, SEQ ID NO:200, SEQ ID NO:201, SEQ ID NO:202, SEQ ID NO:203, SEQ ID NO:204, SEQ ID NO:205, SEQ ID NO:206, SEQ ID NO:207, SEQ ID NO:208, SEQ ID NO:209, SEQ ID NO:210, SEQ ID NO:211, SEQ ID NO:212, SEQ ID NO:213, SEQ ID NO:214, SEQ ID NO:215, SEQ ID NO:216, SEQ ID NO:217, SEQ ID NO:218, SEQ ID NO:219, SEQ ID NO:220, SEQ ID NO:221, SEQ ID NO:222, SEQ ID NO:223, SEQ ID NO:224, SEQ ID NO:225, SEQ ID NO:226, SEQ ID NO:227, SEQ ID NO:228, SEQ ID NO:229, and SEQ ID NO:230. More preferably the Progenipoietin protein is SEQ ID NO:165.

In a preferred embodiment R₂ is selected from the group consisting of G-CSF, G-CSF Ser¹⁷, G-CSF Ala¹⁷ and c-mpl ligand (TPO).

In a preferred embodiment R₂ is an IL-3 variant of SEQ ID 261. In a preferred embodiment R₂ is the IL-3 variant of SEQ ID NO:256, SEQ ID NO:257, SEQ ID NO:258, or SEQ ID NO:259.

In preferred embodiment the linker (L₂) is selected from the group consisting of;

| | |
|---|---|
| Ser; | |
| Asn; | |
| Gly; | |
| Thr; | |
| GlySer; | |
| AlaAla; | |
| GlySerGly; | |
| GlyGlyGly; | |
| GlyAsnGly; | |
| GlyAlaGly; | |
| GlyThrGly; | |
| AlaSerAla; | |
| AlaAlaAla; | |
| GlyGlyGlySer; | SEQ ID NO:231 |
| GlyGlyGlySerGlyGlyGlySer; | SEQ ID NO:232 |
| GlyGlyGlySerGlyGlyGlySerGlyGlyGlySer; | SEQ ID NO:233 |
| SerGlyGlySerGlyGlySer; | SEQ ID NO:234 |
| GluPheGlyAsnMet; | SEQ ID NO:235 |
| GluPheGlyGlyAsnMet; | SEQ ID NO:236 |
| GluPheGlyGlyAsnGlyGlyAsnMet; | SEQ ID NO:237 |
| GlyGlySerAspMetAlaGly; | SEQ ID NO:238 |
| SerGlyGlyAsnGly; | SEQ ID NO:239 |
| SerGlyGlyAsnGlySerGlyGlyAsnGly; | SEQ ID NO:240 |
| SerGlyGlyAsnGlySerGlyGlyAsnGlySerGly | SEQ ID NO:241 |
| GlyAsnGly; | |
| SerGlyGlySerGlySerGlyGlySerGly; | SEQ ID NO:242 |
| SerGlyGlySerGlySerGlyGlySerGlySerGly | SEQ ID NO:243 |
| GlySerGly; | |
| GlyGlyGlySerGlyGly; | SEQ ID NO:244 |
| GlyGlyGlySerGlyGlyGly; | SEQ ID NO:245 |
| GlyGlyGlySerGlyGlyGlySerGlyGly; | SEQ ID NO:246 |
| GlyGlyGlySerGlyGlyGlySerGlyGlyGlySer | SEQ ID NO:247 |
| Gly; | |
| GlyGlyGlySerGlyGlyGlySerGlyGlyGlySer | SEQ ID NO:248 |
| GlyGlyGly; | |
| GlyGlyGlySerGlyGlyGlySerGlyGlyGlySer | SEQ ID NO:249 |
| GlyGlyGlySerGlyGlyGlySerGly; | |
| ProProProTrpSerProArgProLeuGlyAlaThr | SEQ ID NO:250 |
| AlaProThrAlaGlyGlnProProLeu; | |
| ProProProTrpSerProArgProLeuGlyAlaThr | SEQ ID NO:251 |
| AlaProThr; | |
| ValGluThrValPheHisArgValSerGlnAspGly | SEQ ID NO:252 |
| LeuLeuThrSer; | |
| GlyGlyGlySerGlyGlyGlySerGlyGlyGlySer | SEQ ID NO:253 |
| GluGlyGlyGlySerGluGlyGlyGlySerGluGly | |
| GlyGlySerGluGlyGlyGlySerGlyGlyGlySer; | |
| IleSerGluProSerGlyProIleSerThrIleAsn | SEQ ID NO:254 |
| ProSerProProSerLysGluSerHisLysSer | |
| Pro;, | |
| and | |
| IleGluGlyArgIleSerGluProSerGlyProIle | SEQ ID NO:255 |
| SerThrIleAsnProSerProProSerLysGluSer | |
| HisLysSerPro. | |

Any purified and isolated ProGP, which is produced by host cells such as E. coli and animal cells transformed or transfected by using recombinant genetic techniques, may be used in the present invention. Among them, ProGP, which is produced by the transformed E. coli, is particularly preferable. Such ProGP may be obtained in large quantities with high purity and homogeneity. For example, the above ProGP may be prepared according to a method disclosed in WO 98/17810. The term “substantially has the following amino acid sequence” means that the above amino acid sequence may include one or more amino-acid changes (deletion, addition, insertion or replacement) as long as such changes will not cause any disadvantageous non-similarity in function to ProGP. It is more preferable to use the ProGP substantially having an amino acid sequence, in which at least one lysine, aspartic acid, glutamic acid, or unpaired cysteine residue is included.

According to the present invention, poly(ethylene glycol) is covalently bound through amino acid residues of ProGP. The amino acid residue may be any reactive one(s) having, for example, free amino, carboxyl or sulfhydryl (thiol) groups, to which a terminal reactive group of an activated poly(ethylene glycol) may be bound. The amino acid residues having the free amino groups may include lysine residues and/or N-terminal amino acid residue, those having a free carboxyl group may include aspartic acid, glutamic acid and/or C-terminal amino acid residues, and having a sulfhydryl (thiol) such as cysteine.

In another embodiment, oxine chemistries (Lemieux & Bertozzi Tib Tech 16:506-513, 1998) are used to target N-terminal serine residues.

The poly(ethylene glycol) used in the present invention is not restricted to any particular form or molecular weight range. Normally a molecular weight of 500-60,000 is used and preferably of from 1,000-40,000. The poly(ethylene glycol) can also be a branched PEG as described in U.S. Pat. No. 5,932,462, U.S. Pat. No. 5,342,940, U.S. Pat. No. 5,643,575, U.S. Pat. No. 5,919,455, U.S. Pat. No. 6,113,906, and U.S. Pat. No. 5,183,660.

Poly(alkylene oxides), notably poly(ethylene glycol)s, is bound to ProGP via a terminal reactive group, which may or may not leave a linking moiety (spacer) between the PEG and the protein. In order to form the ProGP conjugates of the present invention, polymers such as poly(alkylene oxide) are converted into “activated” forms. The reactive group, for example, is a terminal reactive group, which mediates a bond between chemical moieties on the protein, such as amino, carboxyl or thiol groups, and poly(ethylene glycol). Typically, one or both of the terminal polymer hydroxyl end-groups, (i.e. the alpha and omega terminal hydroxyl groups) are converted into reactive functional groups, which allows covalent conjugation. This process is frequently referred to as “activation” and the poly(ethylene glycol) product having the reactive group is hereinafter referred to as “an activated poly(ethylene glycol)”. Polymers containing both α and ω linking groups are referred to as “bis-activated poly(alkylene oxides)” and are referred to as “bifunctional”. Polymers containing the same reactive group on α and ω terminal hydroxyls are sometimes referred to as “homobifunctional” or “homobis-activated”. Polymers containing different reactive groups on α and ω terminal hydroxyls are sometimes referred to as “heterobifunctional” or “heterobis-activated”. Polymers containing a single reactive group are referred to as “mono-activated” polyalkylene oxides or “mono-functional”. Other substantially non-antigenic polymers are similarly “activated” or “functionalized”.

The activated polymers are thus suitable for mediating a bond between chemical moieties on the protein, such as α-amino, carboxyl or thiol groups, and poly(ethylene glycol). Bis-activated polymers can react in this manner with two protein molecules or one protein molecule and a reactive small molecule in another embodiment to effectively form protein polymers or protein-small molecule conjugates through cross linkages. Functional groups capable of reacting with either the amino terminal α-amino group or ε-amino groups of lysines found on the ProGP include: carbonates such as the p-nitrophenyl, or succinimidyl; carbonyl-imidazole; azlactones; cyclic imide thiones; isocyanates or isothiocyanates and aldehydes. Functional groups capable of reacting with carboxylic acid groups, reactive carbonyl groups and oxidized carbohydrate moieties on ProGP include; primary amines; and hydrazine and hydrazide functional groups such as the acyl hydrazides, carbazates, semicarbamates, thiocarbazates, etc. Mercapto groups, if available on the ProGP, can also be used as attachment sites for suitably activated polymers with reactive groups such as thiols; maleimides, sulfones, and phenyl glyoxals; see, for example, U.S. Pat. No. 5,093,531, the disclosure of which is hereby incorporated by reference. Other nucleophiles capable of reacting with an electrophilic center include, but are not limited to, for example, hydroxyl, amino, carboxyl, thiol, active methylene and the like.

In one preferred embodiment of the invention secondary amine or amide linkages are formed using the ProGP N-terminal amino groups or ε-amino groups of lysine and the activated PEG. In another preferred aspect of the invention, a secondary amine linkage is formed between the N-terminal primary amino group of ProGP and single or branched chain PEG aldehyde by reduction with a suitable reducing agent such as NaCNBH₃, NaBH₃, Pyridine Borane etc. as described in Chamow et al., Bioconjugate Chem. 5: 133-140 (1994) and U.S. Pat. No. 5,824,784.

In another preferred embodiment of the invention, polymers activated with amide-forming linkers such as succinimidyl esters, cyclic imide thiones, or the like are used to effect the linkage between the ProGP and polymer, see for example, U.S. Pat. No. 5,349,001; U.S. Pat. No. 5,405,877; and Greenwald, et al., Crit. Rev. Ther. Drug Carrier Syst. 17:101-161, 2000, which are incorporated herein by reference. One preferred activated poly(ethylene glycol), which may be bound to the free amino groups of ProGP includes single or branched chain N-hydroxysuccinylimide poly(ethylene glycol) may be prepared by activating succinic acid esters of poly(ethylene glycol) with N-hydroxysuccinylimide.

Other preferred embodiments of the invention include using other activated polymers to form covalent linkages of the polymer with the ProGP via ε-amino or other groups. For example, isocyanate or isothiocyanate forms of terminally activated polymers can be used to form urea or thiourea-based linkages with the lysine amino groups.

In another preferred aspect of the invention, carbamate (urethane) linkages are formed with protein amino groups as described in U.S. Pat. Nos. 5,122,614, 5,324,844, and 5,612,640, which are hereby incorporated by reference. Examples include N-succinimidyl carbonate, para-nitrophenyl carbonate, and carbonyl imidazole activated polymers. In another preferred embodiment of this invention, a benzotriazole carbonate derivative of PEG is linked to amino groups on ProGP.

Another aspect of the invention represents a prodrug or sustained release form of ProGP, comprised of a water soluble polymer, such as poly(ethylene glycol), attached to an ProGP molecule by a functional linker that can predictably break down by enzymatic or pH directed hydrolysis to release free ProGP or other ProGP derivative. The prodrug can also be a “double prodrug” (Bundgaard in Advanced Drug Delivery Reviews 3:39-65, 1989) involving the use of a cascade latentiation. In such systems, the hydrolytic reaction involves an initial rate-limiting (slow) enzymatic or pH directed step and a second step involving a rapid non-enzymatic hydrolysis that occurs only after the first has taken place. Such a releasable polymer provides protein conjugates, which are impermanent and could act as a reservoir, that continually discharge ProGP. Such functional linkers are described in U.S. Pat. No. 5,614,549; U.S. Pat. No. 5,840,900; U.S. Pat. No. 5,880,131; U.S. Pat. No. 5,965,119; U.S. Pat. No. 6,011,042; U.S. Pat. No. 6,180,095 B1; Greenwald R. B. et al., J. Med. Chem. 42;3657-3667, 1999; Lee, S. et al., Bioconjugate Chem 12:163-169, 2001; Garman A. J. et al., FEBS Lett. 223:361-365, 1987; Woghiren C. et al., Bioconjucate Chem. 4:314-318, 1993; Roberts M. J. et al., J. Pharm. Sci. 87;1440-1445, 1998; Zhao X., in Ninth Int. Symp. Recent Adv. Drug Delivery Syst. 199; Greenwald R. B. et al., J. Med. Chem. 43:475-487, 2000; and Greenwald R. B. Crit. Rev. Ther. Drug Carrier Syst. 17:101-161, 2000.

Another embodiment of the present invention is a method of conjugating a PEG to a nucleophile where the conjugation is carried out in the presence of an inorganic solvent. A “nucleophile” is defined as proteins including but not limited to antibodies and hematopoietic growth factors, peptides, enzymes, medicinal chemicals or organic moieties. Preferably the inorganic solvent is acetonitrile, methanol, or ethanol. More preferably the inorganic solvent is acetonitrile. Preferably the inorganic concentration is between about 1% and about 25%, more preferably between about 3% and about 13%, and more preferably about 8%.

Conjugation reactions, referred to as pegylation reactions, were historically carried out in solution with molar excess of polymer and without regard to where the polymer will attach to the protein. Such general techniques, however, have typically been proven inadequate for conjugating bioactive proteins to non-antigenic polymers while retaining sufficient bioactivity. One way to maintain the ProGP bioactivity is to substantially avoid the conjugation of those ProGP reactive groups associated with the receptor binding site(s) in the polymer coupling process. Another aspect of the present invention is to provide a process of conjugating poly(ethylene glycol) to ProGP maintaining high levels of retained activity.

The chemical modification through a covalent bond may be performed under any suitable condition generally adopted in a reaction of a biologically active substance with the activated poly(ethylene glycol). The conjugation reaction is carried out under relatively mild conditions to avoid inactivating the ProGP. Mild conditions include maintaining the pH of the reaction solution in the range of 3 to 10 and the reaction temperatures within the range of from about 0°-37° C. In the cases where the reactive amino acid residues in ProGP have free amino groups, the above modification is preferably carried out in a non-limiting list of suitable buffers (pH 3 to 10), including phosphate, citrate, acetate, succinate or HEPES, for 1-48 hrs at 4°-37° C. In targeting N-terminal amino groups with reagents such as PEG aldehydes pH 4-7 is preferably maintained. The activated poly(ethylene glycol) may be used in 0.05-100 times, preferably 0.05-0.5 times, the molar amount of the number of free amino groups of ProGP. On the other hand, where reactive amino acid residues in ProGP have the free carboxyl groups, the above modification is preferably carried out in pH from about 3.5 to about 5.5, for example, the modification with poly(oxyethylenediamine) is carried out in the presence of carbodiimide (pH 4-5) for 1-24 hrs at 4°-37° C. The activated poly(ethylene glycol) may be used in 0.05-300 times the molar amount of the number of free carboxyl groups of ProGP.

In separate embodiments, the upper limit for the amount of polymer included in the conjugation reactions exceeds about 1:1 to the extent that it is possible to react the activated polymer and ProGP without forming a substantial amount of high molecular weight species, i.e. more than about 20% of the conjugates containing more than about one strand of polymer per molecule of ProGP. For example, it is contemplated in this aspect of the invention that ratios of up to about 6:1 can be employed to form significant amounts of the desired conjugates which can thereafter be isolated from any high molecular weight species.

In another aspect of this invention, bifunctionally activated PEG derivatives may be used to generate polymeric ProGP-PEG molecules in which multiple ProGP molecules are crosslinked via PEG. Although the reaction conditions described herein can result in significant amounts of unmodified ProGP, the unmodified ProGP can be readily recycled into future batches for additional conjugation reactions. The processes of the present invention generate surprisingly very little, i.e. less than about 30% and more preferably, less than about 10%, of high molecular weight species and species containing more than one polymer strand per ProGP. These reaction conditions are to be contrasted with those typically used for polymeric conjugation reactions wherein the activated polymer is present in several-fold molar excesses with respect to the target. In other aspects of the invention, the polymer is present in amounts of from about 0.1 to about 50 equivalents per equivalent of ProGP. In other aspects of the invention, the polymer is present in amounts of from about 1 to about 10 equivalents per equivalent of ProGP.

The conjugation reactions of the present invention initially provide a reaction mixture or pool containing mono- and di-PEG-ProGP conjugates, unreacted ProGP, unreacted polymer and usually less than about 20% high molecular weight species. The high molecular weight species include conjugates containing more than one polymer strand and/or polymerized PEG-ProGP species. After the unreacted species and high molecular weight species have been removed, compositions containing primarily mono- and di-polymer-ProGP conjugates are recovered. Given the fact that the conjugates for the most part include a single polymer strand, the conjugates are substantially homogeneous. These modified ProGPs have at least about 5% of the in vitro biological activity associated with the native or unmodified ProGP as measured using standard cell proliferation assays, such as AML, TFl and colony forming unit assays (U.S. Pat. No. 6,030,812 which is incorporated by reference herein). In preferred aspects of the invention, the modified ProGPs have about 25% of the in vitro biological activity, more preferably, the modified ProGPs have about 50% of the in vitro biological activity, more preferably, the modified ProGPs have about 75% of the in vitro biological activity, and most preferably the modified ProGPs have equivalent or improved in vitro biological activity.

The processes of the present invention preferably include rather limited ratios of polymer to ProGP. Thus, the ProGP conjugates have been found to be predominantly limited to species containing only one strand of polymer. Furthermore, the attachment of the polymer to the ProGP reactive groups is substantially less random than when higher molar excesses of polymer linker are used. The unmodified ProGP present in the reaction pool, after the conjugation reaction has been quenched, can be recycled into future reactions using ion exchange or size exclusion chromatography or similar separation techniques.

A poly(ethylene glycol)-modified ProGP, namely chemically modified protein according to the present invention,-may be purified from a reaction mixture by conventional methods which are used for purification of proteins, such as dialysis, salting-out, ultrafiltration, ion-exchange chromatography, gel chromatography and electrophoresis. Ion-exchange chromatography is particularly effective in removing unreacted poly(ethylene glycol) and ProGP. In a further embodiment of the invention, the mono- and di-polymer-ProGP species are isolated from the reaction mixture to remove high molecular weight species, and unmodified ProGP. Separation is effected by placing the mixed species in a buffer solution containing from about 0.5-10 mg/mL of the ProGP-polymer conjugates. Suitable solutions have a pH from about 4 to about 10. The solutions preferably contain one or more buffer salts selected from KCl, NaCl, K₂HPO₄, KH₂PO₄, Na₂HPO₄, NaH₂PO₄, NaHCO₃, NaBO₄, CH₃CO₂H, and NaOH.

Depending upon the reaction buffer, the ProGP polymer conjugate solution may first have to undergo buffer exchange/ultrafiltration to remove any unreacted polymer. For example, the PEG-ProGP conjugate solution can be ultrafiltered across a low molecular weight cut-off (10,000 to 30,000 Dalton) membrane to remove most unwanted materials such as unreacted polymer, surfactants, if present, or the like.

The fractionation of the conjugates into a pool containing the desired species is preferably carried out using an ion exchange chromatography medium. Such media are capable of selectively binding PEG-ProGP conjugates via differences in charge, which vary in a somewhat predictable fashion. For example, the surface charge of ProGP is determined by the number of available charged groups on the surface of the protein. These charged groups typically serve as the point of potential attachment of poly(alkylene oxide) conjugates. Therefore, ProGP conjugates will have a different charge from the other species to allow selective isolation.

Strongly polar anion or cation exchange resins such as quaternary amine or sulfopropyl resins, respectively, are used for the method of the present invention. Cation exchange resins are especially preferred. A non-limiting list of included commercially available cation exchange resins suitable for use with the present invention are SP-hitrap®, SP Sepharose HP® and SP Sepharose® fast flow. Other suitable cation exchange resins e.g. S and CM resins can also be used. A non-limiting list of anion exchange resins, including commercially available anion exchange resins, suitable for use with the present invention are Q-hitrap®, Q Sepharose HP®, and Q sepharose® fast flow. Other suitable anion exchange resins, e.g. DEAE resins, can also be used.

For example, the cation exchange resin is preferably packed in a column and equilibrated by conventional means. A buffer having the same pH and osmolality as the polymer conjugated ProGP solution is used. The elution buffer preferably contains one or more salts selected from KCl, NaCl, K₂HPO₄, KH₂PO₄, Na₂HPO₄, NaH₂PO₄, NaHCO₃, NaBO₄ and (NH₄)₂CO₃. The conjugate-containing solution is then adsorbed onto the column with unreacted polymer and some high molecular weight species not being retained. At the completion of the loading, a gradient flow of an elution buffer with increasing salt concentrations is applied to the column to elute the desired fraction of polyalkylene oxide-conjugated ProGP. The eluted pooled fractions are preferably limited to uniform polymer conjugates after the cation exchange separation step. Any unconjugated ProGP species can then be back washed from the column by conventional techniques. If desired, mono and multiply pegylated ProGP species can be further separated from each other via additional ion exchange chromatography or size exclusion chromatography. Techniques utilizing multiple isocratic steps of increasing concentration can also be used. Multiple isocratic elution steps of increasing concentration will result in the sequential elution of di- and then mono-ProGP-polymer conjugates.

The temperature range for elution is between about 4° C. and about 25° C. Preferably, elution is carried out at a temperature of from about 6° C. to about 22° C. For example, the elution of the PEG-ProGP fraction is detected by UV absorbance at 280 nm. Fraction collection may be achieved through simple time elution profiles.

A surfactant can be used in the processes of conjugating the poly(ethylene glycol) polymer with the ProGP moiety. Suitable surfactants include ionic-type agents such as sodium dodecyl sulfate (SDS). Other ionic surfactants such as lithium dodecyl sulfate, quaternary ammonium compounds, taurocholic acid, caprylic acid, decane sulfonic acid, etc. can also be used. Non-ionic surfactants can also be used. For example, materials such as poly(oxyethylene) sorbitans (Tweens), poly(oxyethylene) ethers (Tritons) can be used. See also Neugebauer, A Guide to the Properties and Uses of Detergents in Biology and Biochemistry (1992) Calbiochem Corp. The only limitations on the surfactants used in the processes of the invention are that they are used under conditions and at concentrations that do not cause substantial irreversible denaturation of the ProGP and do not completely inhibit polymer conjugation. The surfactants are present in the reaction mixtures in amounts from about 0.01-0.5%; preferably from 0.05-0.5%; and most preferably from about 0.075-0.25%. Mixtures of the surfactants are also contemplated.

It is thought that the surfactants provide a temporary, reversible protecting system during the polymer conjugation process. Surfactants have been shown to be effective in selectively discouraging polymer conjugation while allowing lysine-based or amino terminal-based conjugation to proceed.

The present poly(ethylene glycol)-modified ProGP has a more enduring pharmacological effect, which may be possibly attributed to its prolonged half-life in vivo.

An intended use of the PEG-ProGP of the present invention is for the generation of larger numbers of dendritic cells, from precursors, to be used as adjuvants for immunization. Dendritic cells play a crucial role in the immune system. They are the professional antigen-presenting cells most efficient in the activation of resting T cells and are the major antigen-presenting cells for activation of naive T cells in vivo and, thus, for initiation of primary immune responses. They efficiently internalize, process and present soluble tumor-specific antigens (Ag). Dendritic cells have the unique capacity to cluster naive T cells and to respond to Ag encounter by rapid up-regulation of the expression of major histocompatibility complex (MHC) and co-stimulatory molecules, the production of cytokines and migration towards lymphatic organs. Since dendritic cells are of central importance for sensitizing the host against a neoantigen for CD4-dependent immune responses, they may also play a crucial role in the generation and regulation of tumor immunity.

Dendritic cells originate from a bone marrow CD34+ precursor common to granulocytes and macrophages, and the existence of a separate dendritic cell colony-forming unit (CFU-DC) that give rise to pure dendritic cell colonies has been established in humans. In addition, a post-CFU CD14+ intermediate has been described with the potential to differentiate along the dendritic cell or the macrophage pathway under distinct cytokine conditions. This bipotential precursor is present in the bone marrow, cord blood, and peripheral blood. Dendritic cells can be isolated based on specific cell surface markers, such as CD1a+, CD3−, CD4−, CD20−, CD40+, CD80+, and CD83+, to delineate the maturation of cultured dendritic cells.

Dendritic cells based strategies provide a method for enhancing immune response against tumors and infectious agents. AIDS is another disease for which dendritic cell based therapies can be used, since dendritic cells can play a major role in promoting HIV-1 replication. An immunotherapy requires the generation of dendritic cells from cancer patients, their in vitro exposure to tumor Ag, derived from surgically removed tumor masses, and reinjection of these cells into the tumor patients. Relatively crude membrane preparations of tumor cells will suffice as sources of tumor antigen, avoiding the necessity for molecular identification of the tumor antigen. The tumor antigen may also be synthetic peptides, carbohydrates, or nucleic acid sequences. In addition, concomitant administration of cytokines such as the PEG-ProGP of the present invention may further facilitate the induction of tumor immunity. It is foreseen that the immunotherapy can be in an in vivo setting, wherein the PEG-PrOGP of the present invention is administered to a patient, having a tumor, alone or with other hematopoietic growth factors to increase the number of dendritic cells and endogenous tumor antigen is presented on the dendritic cells. It is also envisioned that in vivo immunotherapy can be with exogenous antigen. It is also envisioned that the immunotherapy treatment may include the mobilization of dendritic cell precursors or mature dendritic, by administering the PEG-ProGP of the present invention alone or with other hematopoietic growth factors to the patient, removing the dendritic cell precursors or mature dendritic cells from the patient, exposing the dendritic cells to antigen and returning the dendritic cells to the patient. Furthermore, the dendritic cells that have been removed can be cultured ex vivo with the PEG-ProGP of the present invention alone or with other hematopoietic growth factors to increase the number of dendritic cells prior to exposure to antigen. Dendritic cells based strategies also provide a method for reducing the immune response in autoimmune diseases.

Studies on dendritic cells have been greatly hampered by difficulties in preparing the cells in sufficient numbers and in a reasonably pure form. In an ex-vivo cell expansion setting, granulocyte-macrophage colony-stimulating factor (GM-CSF) and tumor necrosis factor-α (TNF-α) cooperate in the ex vivo generation of dendritic cells from hematopoietic progenitors (CD34+ cells) retrieved from bone marrow, cord blood, or peripheral blood and flk-2/flt-3 ligand and c-kit ligand (stem cell factor [SCF]) synergize to enhance the GM-CSF plus TNF-α induced generation of dendritic cells (Siena, S. et al. Experimental Hematology 23:1463-1471, 1995). Also provide is a method of ex vivo expansion of dendritic cell precursors or mature dendritic cells using the PEG-ProGP of the present invention to provide sufficient quantities of dendritic cells for immunotherapy.

Furthermore, it is observed that the present poly(ethylene glycol)-modified ProGP may accelerate recovery from neutropenia. The present poly(ethylene glycol)-modified ProGP may have essentially the same biological activity as an intact ProGP and may accordingly be used in the same applications. The poly(ethylene glycol)-modified ProGP has an activity for increasing the number of neutrophils, and it is useful therefore in the treatment of general hematopoietic disorders including those arising from chemotherapy or from radiation therapy. It may be also useful in the treatment of infection and in bone marrow transplantation. The modified ProGP of the present invention may be useful in the treatment of diseases characterized by decreased levels of either myeloid, erythroid, lymphoid, or megakaryocyte cells of the hematopoietic system or combinations thereof. In addition, they may be used to activate mature myeloid and/or lymphoid cells. Among conditions susceptible to treatment with the polypeptides of the present invention is leukopenia, a reduction in the number of circulating leukocytes (white cells) in the peripheral blood. Leukopenia may be induced by exposure to certain viruses or to radiation. It is often a side effect of various forms of cancer therapy, e.g., exposure to chemotherapeutic drugs, radiation and of infection or hemorrhage. Therapeutic treatment of leukopenia with these modified ProGP of the present invention may avoid undesirable side effects caused by treatment with presently available drugs.

The modified ProGP of the present invention may be useful in the treatment or prevention of neutropenia and, for example, in the treatment of such conditions as aplastic anemia, cyclic neutropenia, idiopathic neutropenia, Chediak-Higashi syndrome, systemic lupus erythematosus (SLE), leukemia, myelodysplastic syndrome and myelofibrosis.

The modified ProGP of the present invention may be useful in the treatment or prevention of thrombocytopenia. Currently the only therapies for thrombocytopenia are platelet transfusions, which are costly and carry the significant risks of infection (HIV, HBV) and alloimmunization, and IL-11 (Neumega™) that is approved for certain thrombocytopenia. The modified ProGP may alleviate or diminish the need for platelet transfusions. Severe thrombocytopenia may result from genetic defects such as Fanconi's Anemia, Wiscott-Aldrich, or May-Hegglin syndromes. Acquired thrombocytopenia may result from auto- or allo-antibodies as in Immune Thrombocytopenia Purpura, Systemic Lupus Erythematosis, hemolytic anemia, or fetal maternal incompatibility. In addition, splenomegaly, disseminated intravascular coagulation, thrombotic thrombocytopenic purpura, infection, or prosthetic heart valves may result in thrombocytopenia. Severe thrombocytopenia may also result from chemotherapy and/or radiation therapy or cancer. Thrombocytopenia may also result from marrow invasion by carcinoma, lymphoma, leukemia, or fibrosis.

The modified ProGP of the present invention may be useful in the mobilization of hematopoietic progenitors and stem cells into peripheral blood. Peripheral blood derived progenitors have been shown to be effective in reconstituting patients in the setting of autologous marrow transplantation. Hematopoietic growth factors including G-CSF and GM-CSF have been shown to enhance the number of circulating progenitors and stem cells in the peripheral blood. This has simplified the procedure for peripheral stem cell collection and dramatically decreased the cost of the procedure by decreasing the number of phereses required. The modified ProGP may be useful in mobilization of stem cells and further enhance the efficacy of peripheral stem cell transplantation.

Another projected clinical use of growth factors has been in the in vitro activation of hematopoietic progenitors and stem cells for gene therapy. In order to have the gene of interest incorporated into the genome of the hematopoietic progenitor or stem cell one needs to stimulate cell division and DNA replication. Hematopoietic stem cells cycle at a very low frequency, which means that growth factors may be useful to promote gene transduction and thereby enhance the clinical prospects for gene therapy.

Many drugs may cause bone marrow suppression or hematopoietic deficiencies. Examples of such drugs are AZT, DDI, alkylating agents and anti-metabolites used in chemotherapy, antibiotics such as chloramphenicol, penicillin, gancyclovir, daunomycin and sulfa drugs, phenothiazones, tranquilizers such as meprobamate, analgesics such as aminopyrine and dipyrone, anti-convulsants such as phenytoin or carbamazepine, antithyroids such as propylthiouracil and methimazole and diuretics. The modified ProGP of the present invention may be useful in preventing or treating the bone marrow suppression or hematopoietic deficiencies, which often occur in patients treated with these drugs.

Hematopoietic deficiencies may also occur because of viral, microbial, or parasitic infections and as a result of treatment for renal disease or renal failure, e.g., dialysis. The modified ProGP of the present invention may be useful in treating such hematopoietic deficiency.

The treatment of hematopoietic deficiency may include administration of a pharmaceutical composition containing the modified ProGP to a patient. The modified ProGP of the present invention may also be useful for the activation and amplification of hematopoietic precursor cells by treating these cells in vitro with the modified ProGP of the present invention prior to injecting the cells into a patient.

Various immunodeficiencies e.g., in T and/or B-lymphocytes, or immune disorders, e.g., rheumatoid arthritis, may also be beneficially affected by treatment with the modified ProGP of the present invention. Immunodeficiencies may be the result of viral infections e.g. HTLV-I, HTLV-II, HTLV-III, severe exposure to radiation, cancer therapy or the result of other medical treatment. The modified ProGP of the present invention may also be employed, alone or in combination with other hematopoietins, in the treatment of other blood cell deficiencies, including thrombocytopenia (platelet deficiency), or anemia. Other uses for these novel polypeptides are in the treatment of patients recovering from bone marrow transplants in vivo and ex vivo, and in the development of monoclonal and polyclonal antibodies generated by standard methods for diagnostic or therapeutic use.

The present poly(ethylene glycol)-modified ProGP may be formulated into pharmaceuticals containing also a pharmaceutically acceptable diluent, an agent for preparing an isotonic solution, a pH-conditioner and the like in order to administer them into a patient. The above pharmaceuticals may be administered subcutaneously, intramuscularly, intravenously, or orally, depending on a purpose of treatment. A dose may be also based on the kind and condition of the disorder of a patient to be treated, being normally between 0.1 mg and 50 mg by injection and between 0.1 mg and 5 g in an oral administration for an adult

The polymeric substances included are also preferably water-soluble at room temperature. A non-limiting list of such polymers include poly(alkylene oxide) homopolymers such as poly(ethylene glycol) or poly(propylene glycols), poly(oxyethylenated polyols), copolymers thereof and block copolymers thereof, provided that the water solubility of the block copolymers is maintained.

As an alternative to PEG-based polymers, effectively non-antigenic materials such as dextran, poly(vinyl pyrrolidones), poly(acrylamides), poly(vinyl alcohols), carbohydrate-based polymers, and the like can be used. Indeed, the activation of α- and ω-terminal groups of these polymeric substances can be effected in fashions similar to that used to convert poly(alkylene oxides) and thus will be apparent to those of ordinary skill. Those of ordinary skill in the art will realize that the foregoing list is merely illustrative and that all polymer materials having the qualities described herein are contemplated. For purposes of the present invention, “effectively non-antigenic” means all materials understood in the art as being nontoxic and not eliciting an appreciable immunogenic response in mammals.

### DEFINITIONS

The following is a list of abbreviations and the corresponding meanings as used interchangeably herein:

| | |
|---|---|
| g | gram(s) |
| mg | milligram(s) |
| ml or mL | milliliter(s) |
| RT | room temperature |
| PEG | poly (ethylene glycol) |

The complete content of all publications, patents, and patent applications cited in this disclosure are herein incorporated by reference as if each individual publication, patent, or patent application were specifically and individually indicated to be incorporated by reference.

Although the foregoing invention has been described in some detail by way of illustration and example for the purposes of clarity of understanding, it will be readily apparent to one skilled in the art in light of the teachings of this invention that changes and modifications can be made without departing from the spirit and scope of the present invention. The following examples are provided for exemplification purposes only and are not intended to limit the scope of the invention, which has been described in broad terms above.

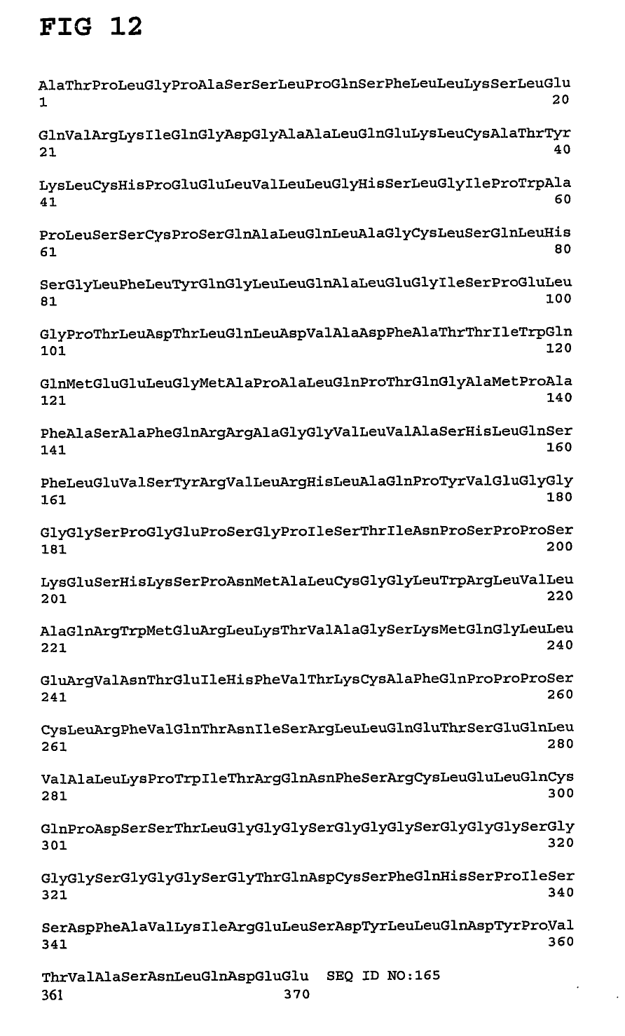

In the following examples, the ProGP polypeptide is that of ProGP-4, the amino acid sequence for which is shown in FIG. 12. It is understood that other members of the ProGP family of polypeptides could also be pegylated in a similar manner as exemplified in the subsequent examples.

### EXAMPLES

### Example 1

Straight Chain 30,000 MW PEG-ProGP 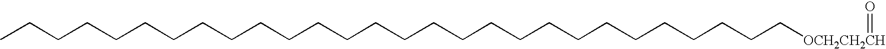
PEG-Aldehyde 5,20 and 30,000 MW

This example demonstrates a method for generation of substantially homogeneous preparations of N-terminally monopegylated ProGP by reductive alkylation. Methoxy-linear PEG-propionaldehyde reagent of approximately 30,000 MW (Shearwater Polymers Inc.) was selectively coupled via reductive amination to the N-terminus of ProGP by taking advantage of the difference in the relative pKₐ value of the primary amine at the N-terminus versus pKₐ values of primary amines at the ε-amino position of lysine residues. ProGP protein dissolved at 1-5 mg/mL in 10 mM sodium Succinate, pH 5.0, or in 10 mM sodium succinate (J. T. Baker, Phillipsburg, N.J.), 8% acetonitrile (Burdick and Jackson, Muskegon, Mich.), pH 5.0, was reacted with Methoxy-PEG-propionaldehyde, M-PEG-ALD, (Shearwater Polymers Inc., Huntsville, Ala.) by addition of solid M-PEG-ALD to yield a relative PEG:ProGP (dimer) molar ratio of 2-12:1. Reactions were catalyzed by addition of stock 1M NaCNBH₄ (Sigma Chemical, St. Louis, Mo.) dissolved in H₂O to a final concentration of 10 mM. Reactions were carried out in the dark at 4° C. for 18-24 hours. Reactions were stopped by addition of 1 M Tris base (Sigma Chemical, St. Louis, Mo.) to a final Tris concentration of 50 mM and a final pH of 8.5.

### Example 2

Straight Chain 20,000 MW PEG-ProGP

Methoxy-linear 20,000 MW PEG-propionaldehyde reagent (Shearwater Polymers Inc.) was coupled to the N-terminus of ProGP using the procedure described for Example 1.

### Example 3

Straight chain 5,000 MW PEG-ProGP

Methoxy-linear 5,000 MW PEG-propionaldehyde reagent (Fluka) was coupled to the N-terminus of ProGP using the procedure described for Example 1.

### Example 4

Branched chain 40,000 MW PEG-ProGP 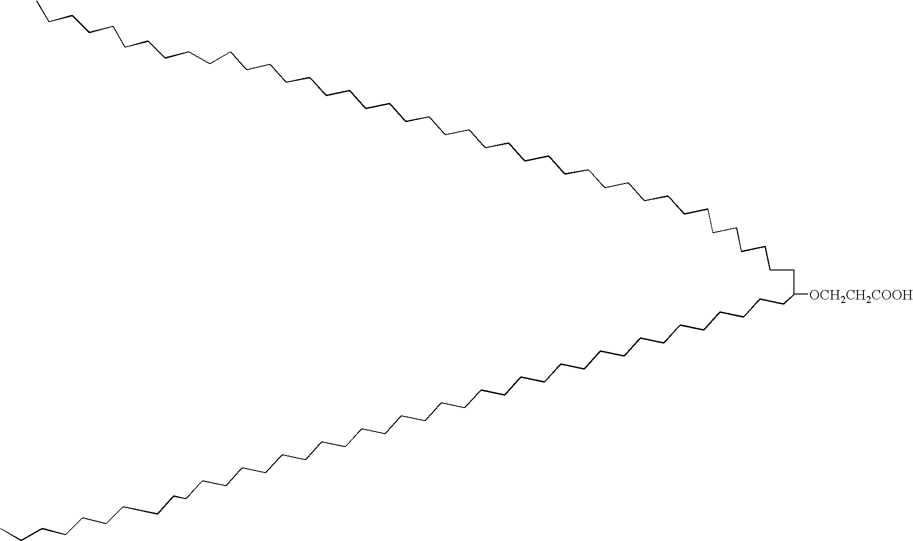
PEG2-ALD 40,000 MW

Methoxy-branched 40,000 MW PEG-propionaldehyde (PEG2-ALD) reagent (Shearwater Polymers Inc.) was coupled to the N-terminus of ProGP using the procedure described for Example 1.

### Example 5

Straight chain 20,000 MW PEG-ProGP 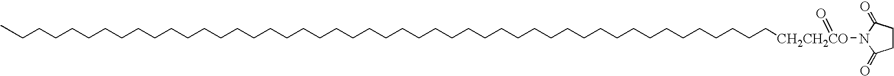
SPA-PEG 5 and 20,000 MW

This example demonstrates a method for generation of substantially homogeneous preparations of monopegylated Progenipoietin (ProGP) using N-hydroxysuccinimidyl (NHS) active esters. ProGP protein stock solution is dissolved at 1-5 mg/mL in 10 mM sodium Succinate, pH 5.0, is titrated to pH 7.2 by addition of 0.25 M HEPES buffer. The solution is then reacted with Methoxy-PEG-succinimidyl propionate (SPA-PEG) by addition of solid SPA-PEG to yield a relative PEG:Progenipoietin molar ratio of 6.5:1. Reactions are carried out at 4° C. for 1 hour. Reactions are stopped by lowering the pH to 4.0 with 0.1 N acetic acid or by adding a SX molar excess of Tris HCl. In a like manner CM-HBA-NHS 5 to 20,000 MW PEG could be used.

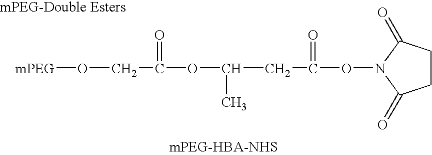

### Example 6

Straight Chain 3,400 MW Biotin-PEG-ProGP 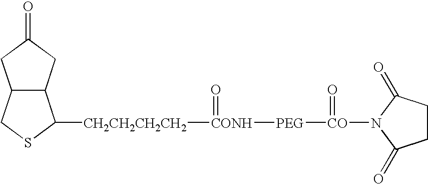
Biotin-PEG-NHS 3,400 MW

3,400 MW Biotin-PEG-CO₂-NHS reagent (Shearwater Polymers Inc.) is coupled to ProGP using the procedure described for Example 5.

### Example 7

Branched 10,000 MW PEG-ProGP 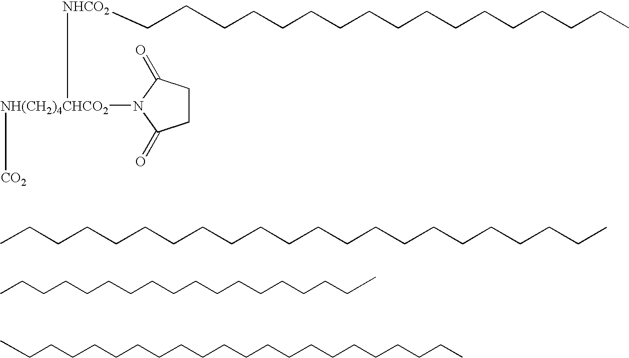
PEG2-NHS 10, 20 and 40,000 MW

10,000 MW branched PEG2-NHS (Shearwater Polymers Inc.) is coupled to ProGP using the procedure described for Example 5.

### Example 8

Branched 20,000 MW PEG-ProGP

20,000 MW branched PEG2-SPA (Shearwater Polymers Inc.) is coupled to ProGP using the procedure described for Example 5.

### Example 9

Branched 40,000 MW PEG-ProGP

40,000 MW branched PEG2-SPA (Shearwater Polymers Inc.) is coupled to ProGP using the procedure described for Example 5.

### Example 10

Straight Chain 20,000 MW PEG-ProGP 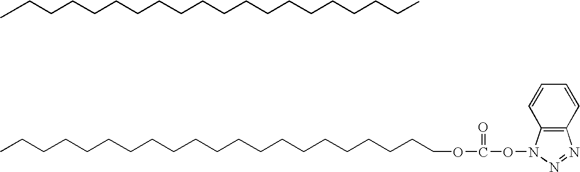
BTC-PEG 20,000 MW

20,000 MW PEG-BTC (Shearwater Polymers Inc.) is coupled to ProGP using the procedure described for Example 4. This example demonstrates a method for generation of substantially homogeneous preparations of pegylated Progenipoietin (ProGP) using benzotriazole carbonate derivatives of PEG.

### Example 11

Straight Chain 5,000 MW PEG-ProGP 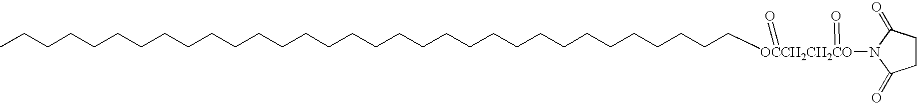
PEG-SS 5,000 MW

5,000 MW succinimidyl succinate-PEG (SS-PEG)(Shearwater Polymers Inc.) is coupled to ProGP using the procedure described for Example 5. This example demonstrates a method for generation of substantially homogeneous preparations of pegylated Progenipoietin (ProGP) using a hydrolyzable linkage.

### Example 12

Straight Chain 20,000 MW PEG-ProGP 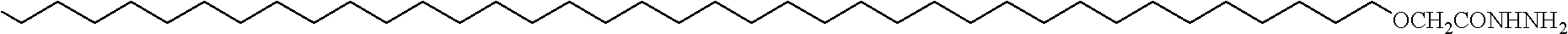
PEG-Hydrazide 20,000 MW

This example demonstrates a method for generation of substantially homogeneous preparations of pegylated Progenipoietin (ProGP) using 20,000 MW methoxy-PEG-hydrazide, HZ-PEG (Shearwater Polymers Inc.). ProGP protein stock solution is dissolved at 1-5 mg/mL in 10 mM sodium Succinate, pH 5.0. The solution is then reacted with HZ-PEG by addition of solid to yield a relative PEG:Progenipoietin molar ratio of 6.5-26:1 reactions are catalyzed with carbodiimide (EDC, EOAC) at a final concentration of 2 mM. Reactions are carried out at 4° C. for 2 hours. Reactions are stopped by lowering the pH to 4 with 0.1 N acetic acid.

### Examples 13

Multi-pegylated Species

Modified ProGPs having two or more PEGs (multi-pegylated) attached were also obtained from Example 1-4 and were separated from the mono-pegylated species using anion exchange chromatography. Modified ProGPs having two or more PEGs (multi-pegylated) attached are also separated from mono-PEGylated species using cation exchange chromatography.

Modified ProGPs having two or more PEGs (multi-pegylated) attached can also be obtained from examples 5-13 and can be purified in similar fashion to examples 1-4.

### Example 14

Purification of Pegylated ProGP

Pegylated ProGP species were purified from the reaction mixture to >95% (SEC analysis) using a single ion exchange chromatography step

Anion Exchange Chromatography

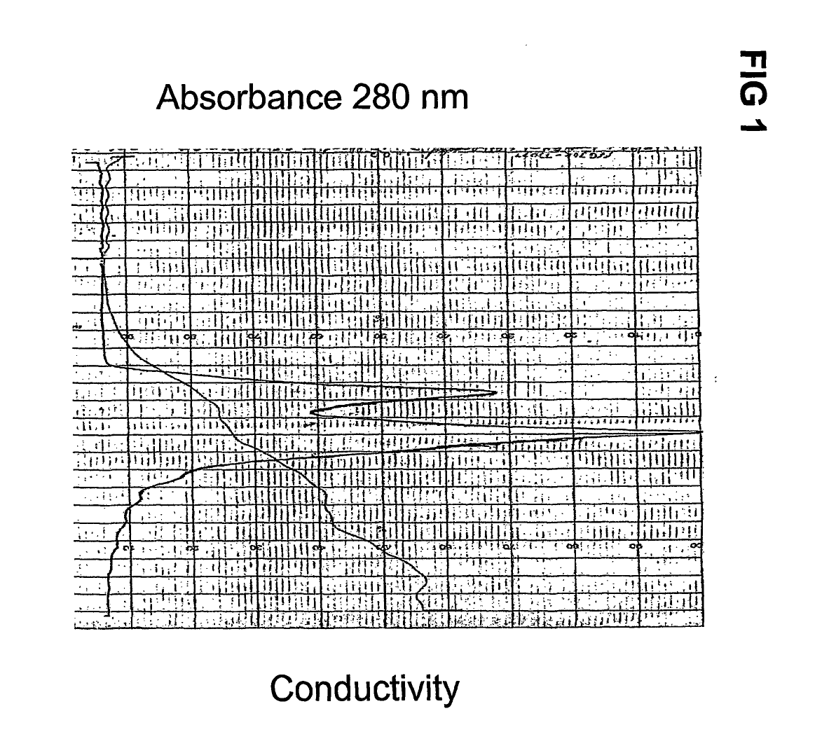

Mono-Pegylated 30K 20K and 5K PEG aldehyde ProGP species were purified from the reaction mixture to >95% (SEC analysis) using a single anion exchange chromatography step. Mono-Pegylated ProGP was purified from unmodified ProGP and multi-PEGylated ProGP species using anion exchange chromatography (FIG. 1). A typical 30K aldehyde ProGP reaction mixture (50-350 mg protein), as described above, was purified on a Q-Sepharose High Performance column (XK 26/20, 70 ml bed volume, Amersham Pharmacia Biotech, Piscataway, N.J.) equilibrated in 50 mM Tris, pH 8.5 (Buffer A). The reaction mixture was diluted 5× with 50 mM Tris, 8% Acetonitrile, pH 8.5 and loaded onto the column at a flow rate of 10 mL/min. The column was washed with 5 column volumes of buffer A, followed by 5 column volumes of buffer A containing 30 mM NaCl. Subsequently, the various ProGP species were eluted from the column in 20 column volumes of Buffer A and a linear NaCl gradient of 30-130 mM. The eluant was monitored by absorbance at 280 nm (A₂₈₀) and 12 mL fractions were collected. Fractions were pooled as to extent of pegylation, e.g., mono, di, tri etc. (as assessed in example 15). The pH of the pool was lowered to 7.5 using 1M HCl, and the pool was then concentrated to 1-5 mg/mL in an 10 K Omegacell stirred cell concentrator (Filtron Technology Corporation, Northborough, Mass.). Protein concentration of pool was determined by A₂₈₀ using an extinction coefficient of 0.97%. Total yield of purified mono 30 K PEG-aldehyde ProGP from this process was 25-30%.

Cation Exchange Chromatography

Cation exchange chromatography is carried out on an SP Sepharose high performance column (Pharmacia XK 26/20, 70 ml bed volume) equilibrated in 10 mM sodium acetate pH 4.5 (Buffer C). The reaction mixture is diluted 10× with buffer C and loaded onto the column at a flow rate of 5 mL/min. Next the column is washed with 5 column volumes of buffer C, followed by 5 column volumes of 12% buffer D (10 mM acetate pH 4.5, 1 M NaCl). Subsequently, the PEG-ProGP species are eluted from the column with a linear gradient of 12 to 27% buffer D in 20 column volumes. The eluant is monitored at 280 nm and 10 mn fractions are collected. Fractions are pooled according to extent of pegylation (mono, di, tri etc.), exchanged into 10 mM acetate pH 4.5 buffer and concentrated to 1-5 mg/mL in a stirred cell fitted with an Amicon YM10 membrane. Protein concentration of pool was determined by A280 nm using an extinction coefficient of 0.71. Total yield of monopegylated ProGP from this process is 10 to 50%.

### Example 15

### Biochemical Characterization

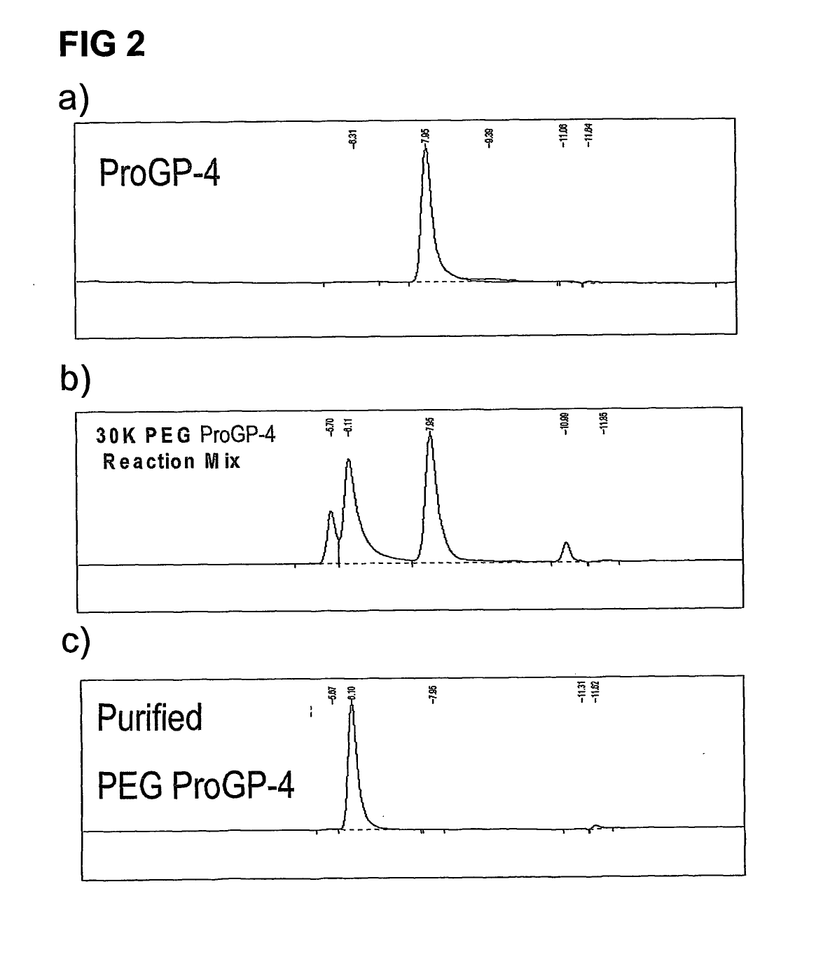

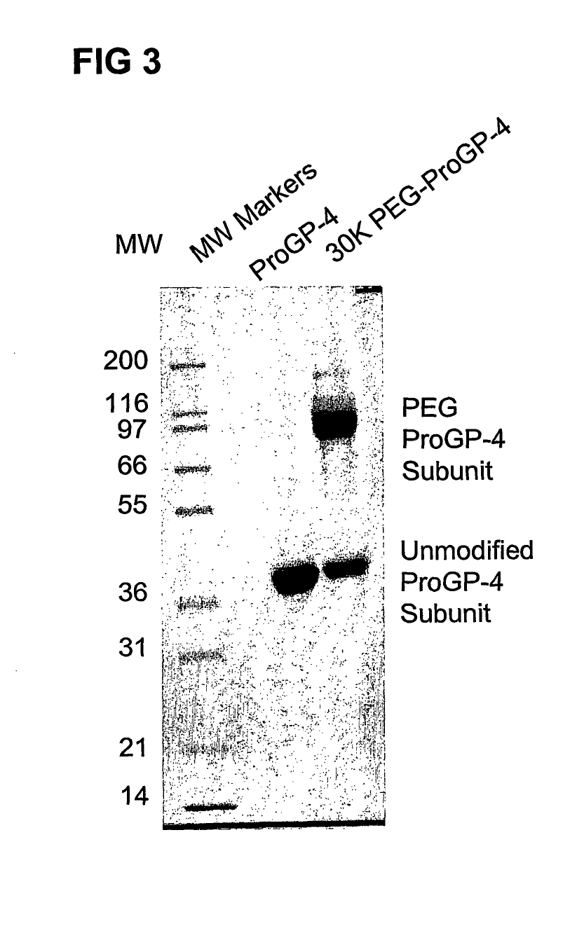

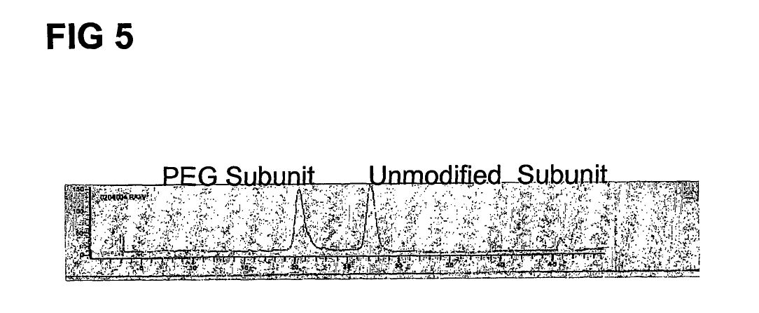

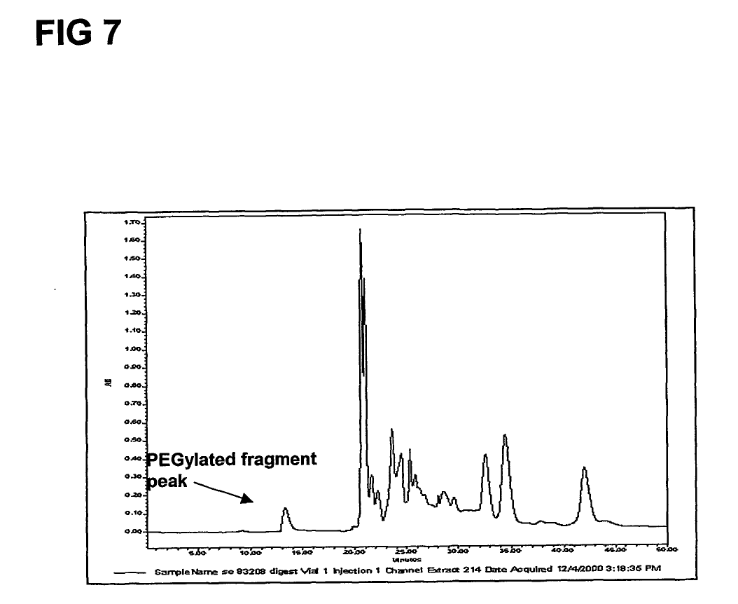

The purified pegylated ProGP pools were characterized by SDS-PAGE (FIG. 3), Non denaturing and denaturing Size Exclusion Chromatography (FIG. 2,4), RP HPLC (FIG. 5), Tryptic mapping (FIG. 7), and MALDI-TOF 6).

Size Exclusion High Performance Liquid Chromatography (SEC-HPLC)

Non-denaturing SEC-HPLC

The reaction of 30K Methoxy-PEG propionaldehyde with ProGP, anion exchange purification, and final purified products were assessed using non-denaturing SEC-HPLC (FIG. 2a, 2b). Analytical non-denaturing SEC-HPLC was carried out using a Superdex 200 HR 10/30 column (Amersham Pharmacia Biotech, Piscataway, N.J.) in 50 mM Tris pH 7.5, 150 mM NaCl at a flow rate of 0.4 mL/minute.

PEGylation greatly increases the hydrodynamic volume of the protein resulting in a shift to an earlier retention time. Two PEGylated species were observed in the 30K PEG aldehyde ProGP reaction mixture along with unmodified ProGP. These PEGylated and non-PEGylated species were separated on Q-Sepharose chromatography (FIG. 1), and the resultant purified mono 30K PEG-Aldehyde ProGP) was subsequently shown to elute as a single peak on non-denaturing SEC (>95% purity, FIG. 2c). The Q-Sepharose chromatography step effectively removed free PEG, non-PEGylated ProGP dimer and multi PEGylated species from the mono-Pegylated ProGP.

Denaturing SDS SEC-HPLC

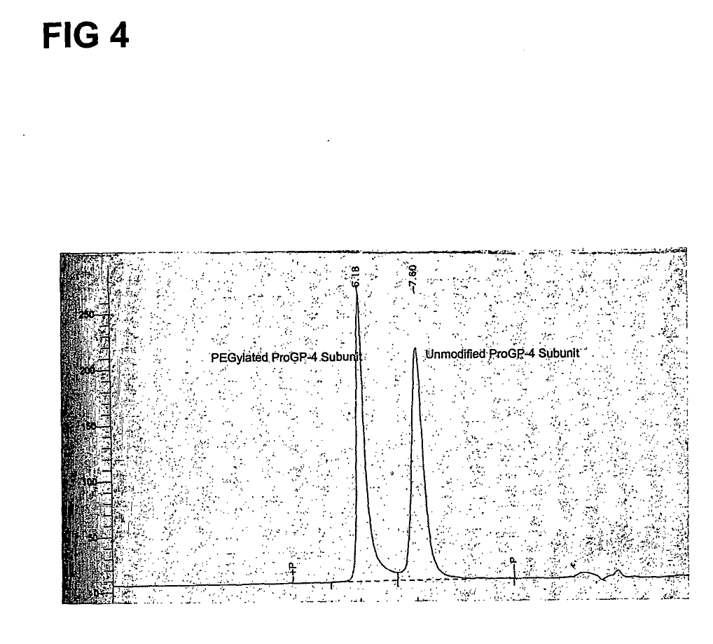

Denaturing SDS SEC, which disassociates the non-covalent ProGP dimer, was used to demonstrate that mono 30K PEG aldehyde ProGP is only PEGylated on one subunit (monomer) of the ProGP dimer (FIG. 4). Denaturing SEC-HPLC was carried out in the same fashion as described for non-denaturing SEC-HPLC with the exception that 0.1% SDS was included in the running buffer. Protein elution was followed by monitoring the absorbance at 220 nm. Purified mono 30K PEG aldehyde ProGP, which elutes as a single peak on non-denaturing SEC, is separated into a PEGylated subunit and a non-PEGylated subunit (as identified below) under denaturing conditions.

SDS PAGE

SDS-PAGE which disassociates the non-covalent ProGP dimer, was used to demonstrate purity and that mono 30K PEG aldehyde ProGP is only PEGylated on one subunit (monomer) of the ProGP dimer (FIG. 3). SDS-PAGE was carried out on 1 mm thick 12% Tris glycine gels (Invitrogen, Carlsbad, Calif.) under reducing and non-reducing conditions and stained using a Novex Colloidal Coomassie™ G-250 staining kit gels (Invitrogen, Carlsbad, Calif.). Purified mono-30K PEG aldehyde ProGP, which elutes as a single peak on non-denaturing SEC, is separated into a PEGylated subunit and a non-PEGylated subunit.

Reversed Phase HPLC (RP HPLC)

RP HPLC was carried out on a Phenomenex Jupiter C₁₈ column (4.6×250 mm, 5 μm particle size) at a temperature of 50° C. samples were loaded onto the column equilibrated in 40% acetonitrile, 0.1% TFA at 1 mL/min. The column was washed with 3 mL 58% acetonitrile. Subsequently, the protein was eluted with a gradient from 58 to 63% acetonitrile over 27 minutes. Preparative and analytical RP-HPLC was carried out on a Jupiter C₁₈ column, 4.6×250 mm, 5mm particle size, (Phenomenex, Torrance, Calif.) at a temperature of 50° C. Samples were loaded onto the column equilibrated in 40% acetonitrile, 0.1% TFA at 1 mL/min. The column was washed with 3 mL 58% acetonitrile. Subsequently, the protein was eluted with a gradient from 58 to 63% acetonitrile over 27 minutes. The eluent was monitored for absorbance at 214 nm. Purified PEG-ProGP, which elutes as a single peak on non-denaturing SEC, is separated (FIG. 5) into a PEGylated subunit and a non-PEGylated subunit (as identified below) under denaturing conditions. In order to confirm the identity of each species, peaks from RP-HPLC elution (FIG. 5) were collected for MALDI-TOF MS and N-terminal sequencing experiments.

N-terminal Sequence and Peptide Mapping

Automated Edman degradation chemistry was used to determine the NH2-terminal protein sequence (SOP 400.324). An Applied Biosystems Model 494 Procise sequencer (Perkin Elmer, Wellesley, Mass.) was employed for the degradation. The respective PTH-AA derivatives were identified by RP-HPLC analysis in an on-line fashion employing an Applied Biosystems Model 140C PTH analyzer fitted with a Perkin Elmer/Brownlee 2.1 mm i.d. PTH-C18 column. N-terminal sequencing of the RP-HPLC peak (FIG. 5) corresponding to the correct mass for PEG-ProGP resulted in two signals. A major signal (approximately 75% yield) had the expected sequence for PEG-ProGP except for the absence of the N-terminal amino acid. This result is as expected for a N-terminally PEGylated protein. The residue of the first cycle is unrecoverable due to the attached PEG moiety. A minor signal (approximately 25% yield) had the correct N-terminal amino acid sequence. Considering that the peak collected off the RP-HPLC is 100% PEGylated, these data suggest that 75% of the PEG modification is at the N-terminus with remainder apparently linked to one of several possible lysine residues.

Tryptic Digest

PEGylated ProGP was digested at 1 mg/mL with trypsin (Promega) Dulbecco's modified PBS (Pierce), 10 mM Tris, pH 7.5 (Sigma) Trypsin at 50:1, reconstituted in 40% ACN (B&J), 0.1% TFA (Pierce). Following digestion overnight at 37°, a second aliquot of trypsin was added and the solution again digested overnight at 37°. Digestion was terminated with 5% 1.0 N HCl (Fisher), and, when necessary, the digest was stored at 4° until use. 175 μg was injected onto a TosoHaas 3000PW column equilibrated in 35% ACN, 0.1% TFA at 0.5 mL/min, and eluted over 50 minutes (FIG. 7). Fractions were collected manually from 0 to 25 minutes. The fraction containing PEGylated fragments were sequenced by Edman chemistry. The results indicated that about 77% of the PEG conjugation occurred at the alpha amino group on the N-terminal alanine, with the remaining 23% distributed as 14% at K284, 5% at K201, and 4% at K252. Matrix Assisted Laser Desorption Ionization Time of Flight Mass Spectrometry (MALDI-TOF MS)

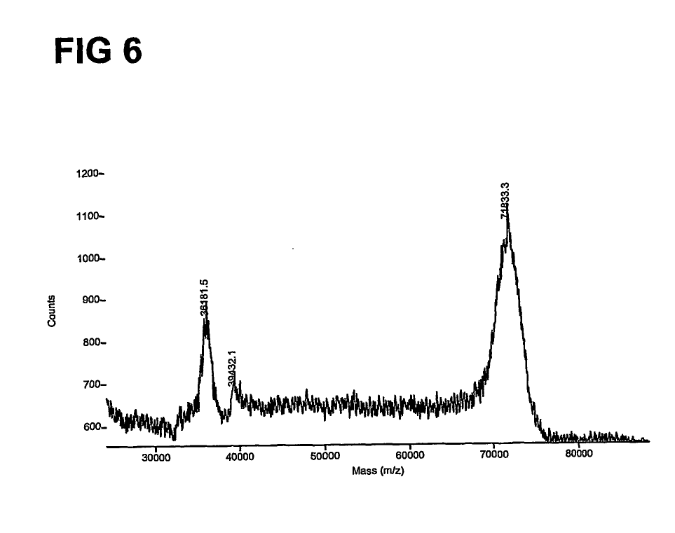

RP-HPLC purified PEGylated, non-PEGylated monomers of ProGP were concentrated, and approximately 1 μL of the concentrate was spotted onto a MALDI target with 3,5-dimethoxy-4-hydroxy-cinnamic acid as the matrix. Spectra were obtained using a Perseptive Biosystems Voyager MALDI-TOF (Perseptive Biosystems,)by monitoring positive ions in the linear mode. One hundred twenty three scans were collected and averaged. Masses were calculated using BSA as a standard. MALDI-TOF MS confirmed the correct masses for each subunit as mono-30K PEGylated-ProGP-4 (PEG-ProGP) monomer (FIG. 6) and non-PEGylated ProGP-4 monomer (data not shown). The early eluting peak had a molecular mass of 71,000 kDa corresponding to 32,000 kDa PEG plus 39,000 kDa ProGP-4. The late eluting peak had correct the molecular mass (39,000 kDa ) for ProGP-4.

### Example 16

BAF3/G-CSFR Cell Proliferation Assay

Mouse BaF3 cell lines transfected with genes encoding the human G-CSF receptor (mBaF3/hG-CSFR) were used to examine hG-CSF agonist activity. mBaF3/hG-CSFR cells were seeded at 2.5×10⁴ cells/well in 96 well microtiter plates containing serial dilution of ProGP and PEGylated ProGP. Cells were pulsed at T₅₆ hours with [methyl-³H]-thymidine at 0.5 mCi per well for 18 hours. Plates were harvested onto glass fiber filter mats, and the incorporated radioactivity was measured by scintillation spectroscopy. The assay medium for the cell lines consisted of IMDM supplemented with bovine serum albumin (BSA) (Boehringer Mannheim, Indianapolis, Ind.), 500 μg/ml, human transferrin (Sigma, St. Louis, Mo.), 100 μg/ml, a lipid substitute consisting of 2.5 mg of phosphatidyl choline/ml of BSA and 50 mM 2-mercaptoethanol. The G-CSF receptor agonist activity of PEG-ProGP was enhanced when compared to ProGP-4 (Table 1). EC₅₀ value for PEG-ProGP (0.005 nM) was approximately 4 fold lower (p<0.05) than that determined for ProGP-4 (0.021 nM).

### Example 17

BAF3/Flt3 Cell Proliferation Assay

A chimeric Flt3/G-CSF receptor molecule was constructed using the extracellular and transmembrane domains of human Flt3, a human IL-3 leader sequence, and the cytoplasmic domain of human G-CSF receptor. The resulting construct was used to transfect the mouse lymphoid cell line Baf/3. The protocol described produced stable clones of Baf/3 that proliferated in response to human FL but not to other human cytokines tested. This clonal line was used to analyze the Flt3 agonist activity of ProGP and pegylated ProGP. Cells were seeded at 2.5×10⁴ cells/well in 96 well microtiter plates containing serial dilutions of each growth factor in serum free IMDM, human transferrin (100 μg/ml), lipid substitute of 2.5 mg phosphatidyl choline/ml bovine serum albumin (500 μg/ml), and 2-mercaptoethanol (50 m). After 56 hr, cells were pulsed with methyl ³H thymidine at 0.5 μCi per well for 14-18 hr, harvested and incorporated radioactivity was measured by scintillation spectroscopy. The PEGylated forms of ProGP maintained the biological activity of ProGP-4 (Table 1).

TABLE 1
Comparison of ProGP-4 and 30K PEG ALD ProGP-4
(Example 1) Induced Cell Proliferation

| | BAF3/Flt | | BAF3/G-CSF | |
|---|---|---|---|---|
| | Average | Average | Average | Average |
| | EC₅₀ | Rel. | EC₅₀ | Rel. |
| | (n = 12) | Potency | (n = 9) | Potency |
| r-hG- | >200 | n/a | 0.025 ± | 1.00 |
| CSF | | | 0.010 | |
| r-hFlt3 | 0.03 ± | 1.00 | >2.0 | n/a |
| | 0.03 | | | |
| ProGP | 0.25 ± | 0.16 ± | 0.021 ± | 1.16 ± |
| | 0.17† | 0.17 | 0.013† | 0.98 |
| Peg | 0.63 ± | 0.05 ± | 0.005 ± | 8.37 ± |
| ProGP-4 | 0.48† | 0.03 | 0.003† | 5.78 |

### Example 18

CFU-GM Clonogenic Assays

Expansion of hematopoietic progenitors was demonstrated using human bone marrow -derived CD34+ cells in a colony forming unit granulocyte/macrophage (CFU-GM) assay, where clonogenic progenitors divide and differentiate in a semi-solid media in response to growth factors. Fresh bone marrow (BM) aspirates were obtained through a collaboration with the St. Louis University Medical School. Mononuclear cell fractions were recovered following density gradient centrifugation with Ficoll-Hypaque. CD34⁺ (stem and progenitor) cells were subsequently isolated by positive selection using the Isolex 50 stem cell reagent kit (Baxter Healthcare Corporation, Deerfield, Ill.). This procedure yields an enriched cellular product where >90% of the cells express the CD34+ cell surface antigen. These CD34⁺ cells were seeded in 35 mm tissue culture plates (10,000 cells/dish) in MethoCult H4230 (StemCell Technologies, Vancouver, BC) containing 0.9% Methylcellulose in Iscove's modified Dulbecco's medium (IMDM), 30% FBS, 1% BSA, 1 mM 2-mercaptoethanol and 2 mM L-glutamine.

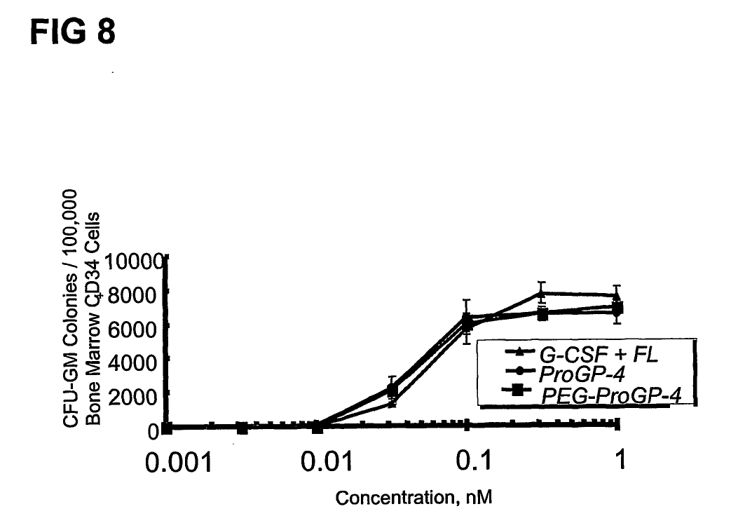

Cultures were incubated with growth factors for 10-12 days at 37° C. in humidified air containing 5% CO₂. The concentrations of the respective receptor agonists in co-addition experiments are equimolar each at the indicated concentration value. Hematopoietic colonies (>50 cells) were counted using an inverted microscope. The Peg ProGP induced differentiation and expansion of hematopoietic progenitor cells into colony forming unit granulocyte/macrophage cells (CFU-GM) was equal to the response elicited from ProGP-4 or co-administration of human flt3 ligand and hG-CSF (FIG. 8).

### Example 19

Growth Factor Administration For Pharmacokinetics (PK)

Female C57BL/6 (8-12 wk old, Charles River, Raleigh, N.C.) mice were housed in the Pharmacia animal facility. ProGP and Pegylated ProGP were diluted in PBS (Life Technologies, Grand Island, N.Y.) and administered to mice at a dose of 100 μg/injection. Mice were injected a single subcutaneous (SC) dose.

ProGP ELISA

ProGP and pegylated ProGP protein concentration levels in mouse plasma were determined using a sandwich ELISA. 96-well microtiter plates were coated with 150 ml/well affinity purified goat-anti-Flt3 ligand polyclonal diluted to 1 μg/ml in 100 mM NaHCO₃, pH 8.2. Plates were incubated overnight at room temperature in a humidified chamber and blocked for one hour at 37° C. with phosphate buffered saline, containing 3 BSA and 0.05% Polyoxyethylene-Sorbitan Monolaurate (Tween 20), pH 7.4. Plates were washed four times with 150 mM NaCl containing 0.05% Tween 20 (wash buffer). Plasma PK samples were diluted in assay buffer (PBS, 0.1% BSA, 0.01% Tween 20), pH 7.4 and titered 1:2 in an assay matrix of mouse pooled plasma. The plasma concentration of the matrix and the samples were matched by percentage. Plates were incubated for 2.5 hours at 37° C. in a humidified chamber, then washed 4 times with wash buffer. Affinity purified goat-anti-human G-CSF receptor agonist polyclonal antibody conjugated to horse radish peroxidase was diluted 1:10000 (0.25-0.05 μg/mL) in assay buffer and 150 μl were added to each well. Plates were incubated for 1.5 hours at 37° C. in a humidified chamber. Wells were emptied and each well was again washed four times with wash buffer. Each well then received 150 mL of TMB peroxidase substrate solution. Plates were incubated at room temperature for about 10 minutes and read at a test wavelength of 650 nm on a microtiter plate reader (Molecular Devices Corporation). Concentrations of immuno-reactive ProGP in unknown PK samples were calculated from a standard curve of either ProGP-4 or PEG-ProGP using a four parameter curve fitting program supplied by Molecular Devices.

Pharmacokinetic Data Analysis

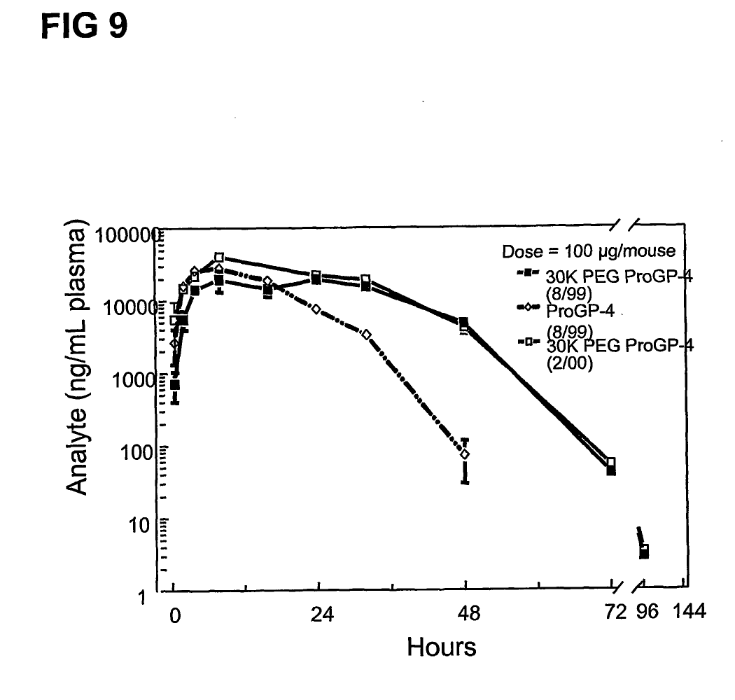

PK parameters reported in Table 2 were determined by noncompartmental analysis using WinNonlin (version 3.0, Pharsight, Chapel Hill, N.C.). Plasma concentration data (n=3) were averaged and a single PK analysis was performed on the mean. The apparent terminal half life (t_{1/2}) was manually selected during non-compartmental analysis by visual inspection of log-linear plot of plasma concentration vs. time. A comparison of the pharmacokinetics of ProGP-4 and PEG-ProGP are shown in FIG. 9 and Table 2. The data show that a single 100 μg dose of PEG-ProGP in mice, administered subcutaneously, has a protracted plasma residency time versus that of ProGP-4. At 48 hours the concentration of PEG-ProGP in the plasma was approximately 70 fold higher than ProGP-4. Detectable levels of PEG-ProGP were observed 96 hours after dosing while ProGP-4 was not detectable at 72 hours. PK values such as terminal half life (t_{1/2}), clearance (Cl), time to maximum concentration (Tmax) and maximum concentration (Cmax) were not significantly different for PEG-ProGP and ProGP-4. In summary, PEGylation extends the time at which the protein circulates in the blood in mice.

TABLE 2
Comparison of ProGP-4 and PEG ProGP-4
(Example 1) Pharmacokinetics in mice

| | Mice | Dose | | | AUC | | | | |
|---|---|---|---|---|---|---|---|---|---|
| | (n) | (μg/mouse) | T*max | C*max | (I) | CL/F | Vd/F | t_{1/2} | C_{48 hr} |
| ProGP | 3 | 100 | 8 | 28,000 | 529 | 0.189 | 0.944 | 3.46 | 72 |
| PEG | 3 | 100 | (8) | 19,500 | 720 | 0.139 | 0.977 | 4.87 | 4744 |
| ProGP-4 | | | 24^{a} | | | | | | |
| PEG | 3 | 100 | 8 | 40,500 | 1080 | 0.0926 | 0.657 | 4.92 | 4181 |
| ProGP-4 | | | | | | | | | |

Growth Factor Administration for Pharmacodynamics (PD)

Female C57BL/6 (8-12 wk old, Charles River, Raleigh, N.C.) mice were housed in the Pharmacia animal facility. ProGP-4 and PEG-ProGP were diluted in PBS (Life Technologies, Grand Island, N.Y.) and administered to mice at doses ranging between 100 to 500 μg/injection. Mice were injected subcutaneous (SC) as follows: ProGP-4 dosed daily either on days 0-6 or days 0-9 (0.1 mg/injection); PEG-ProGP dosed either on days 0, 3 and 6 or 0, 3, 6 and 9 (0.3 mg /injection); PEG-ProGP dosed on days 0 or days 0 and 5 (0.5 mg/injection). Vehicle controls dosed daily on days 0-9 with PBS (0.1 ml/injection).

Peripheral Blood and Spleen Processing

Peripheral blood was collected from CO₂-anesthetized mice into heparin-coated tubes by cardiac puncture. Spleens were harvested from euthanized animals immediately following blood collection. A total white blood cell (WBC) count was done on whole blood using a cell counter (Coulter ZM, Miami Lakes, Fla.). Erythrocytes were lysed using a 0.15 M hypotonic ammonium chloride solution (Stem Cell Technology, Vancouver, BC). Cells were collected by centrifugation (300×g for 10 min) and washed with cold IMDM (Life Technologies, Grand Island, N.Y.) containing 2% FBS (IMDM-FBS, Bioproducts for Science, Indianapolis, Ind.). Spleens were collected and processed in cold IMDM-FBS, homogenized into a cell suspension and filtered through a 70 um nylon mesh. Erythrocytes were lysed using hypotonic ammonium chloride solution, spleen cells were washed with cold IMDM-FBS and filtered through a 70 um mesh. Spleen cells were resuspended in 25 ml of IMDM buffer containing 2% BSA and counted to determine total number of splenocytes/spleen.

Flow Cytometry Reagents

Fluorochrome- or biotin-conjugated monoclonal antibodies were purchased from Pharmingen (San Diego, Calif.). CD16/CD32 (Fc II/III receptor antibody, Fc Block), FITC-conjugated antibodies CD11c/HL3 and PE-conjugated antibodies MHC Class II (I-A^{b/}/AF6-120.1) were used at 1-5 μg/ml. Isotype matched controls were used for the different antibody combinations.

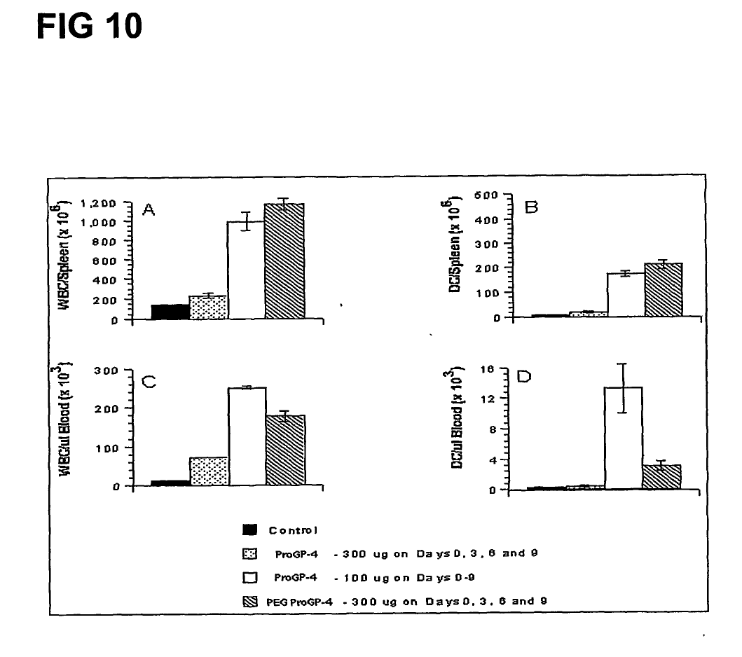

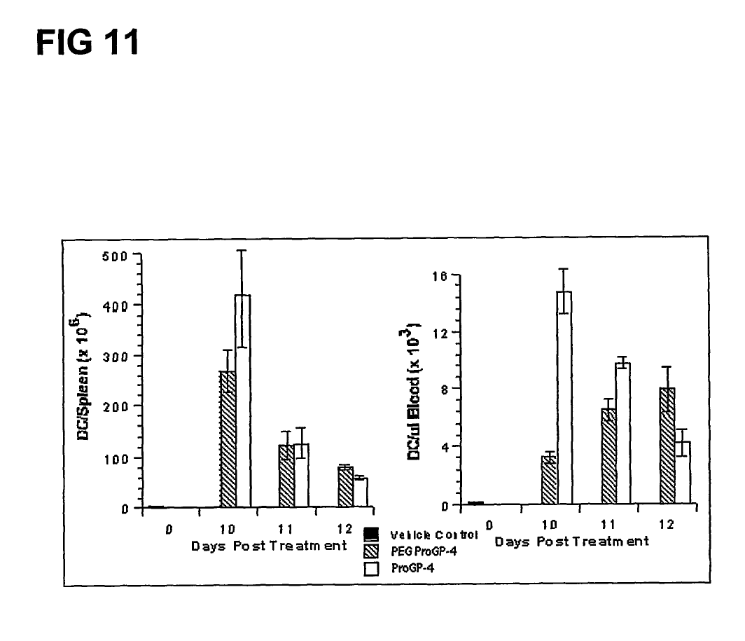

The total white blood cell (WBC) and dendritic cell (DC) responses in the spleen were similar for PEG-ProGP dosed every third day compared to ProGP-4 dosed daily (FIG. 10) The relative DC response in the peripheral blood at day 10 was greater for PEG-ProGP dosed every third day compared to ProGP-4 dosed every day. DC mobilization kinetics (FIG. 11) are similar for ProGP-4 and PEG-ProGP-4 (Example 1) in the spleen; whereas, in the peripheral blood the DC mobilization kinetics are modified for PEG-ProGP-4 (Example 1) dosed every third day when compared to ProGP-4 dosed every day.

What is claimed is:

1. A Progenipoietin conjugate having at least one water-soluble polymer molecule covalently attached to at least one amino acid residue of a biologically active Progenipoietin polypeptide.

2. The Progenipoietin conjugate of claim 1 wherein said polymer is a poly(ethylene oxide) molecule.

3. The Progenipoietin conjugate of claim 2 wherein said poly(ethylene oxide) molecule is a poly(ethylene glycol) molecule.

4. The Progenipoietin conjugate of claim 3 wherein the poly(ethylene glycol) is attached at an amino acid residue having a free amino, carboxyl or sulfhydryl group(s).

5. The Progenipoietin conjugate of claim 4 wherein said poly(ethylene glycol) is conjugated through a activated poly(ethylene glycol).

6. The Progenipoietin conjugate of claim 5 wherein said activated poly(ethylene glycol) is selected from the group consisting of, para-nitrophenyl, succinimidyl, carbonyl imidazole, azlactones, cyclic imide thiones, isocyanates, isothiocyanates, aldehydes, primary amines, hydrazine, acyl hydrazides, carbazates, semicarbamates, thiocarbazates, thiols, maleimides, sulfones, and phenyl glyoxals.

7. The Progenipoietin conjugate of claim 6 wherein said activated poly(ethylene glycol) is selected from the group consisting of, succinimidyl, carbonyl imidazole, aldehydes, acyl hydrazides, carbazates, semicarbamates, and maleimides.

8. The Progenipoietin conjugate of claim 7 wherein said poly(ethylene glycol) has a molecular weight of between about 0.5 kDa and about 100 kDa.

9. The Progenipoietin conjugate of claim 8 wherein said poly(ethylene glycol) has a molecular weight of between about 3.4 kDa and about 40 kDa.

10. The Progenipoietin conjugate of claim 4 wherein said poly(ethylene glycol) is a branched polymer.

11. The Progenipoietin conjugate of claim 10 wherein branched poly(ethylene glycol) polymer has a molecular weight of between about 10 kDa and about 40 kDa.

12. The Progenipoietin conjugate of claim 4 wherein said poly(ethylene glycol) is a bifunctional polymer.

13. The Progenipoietin conjugate of claim 1, 2, 3, 4, 5, 6, 7, 8, 9, 10, 11, or 12 wherein said Progenipoietin polypeptide is of the formula:
R₁-L₁-R₂, R₂-L₁-R₁, R₁-R₂, or R₂-R₁
wherein R₁ is a polypeptide comprising; a modified flt-3 ligand amino acid sequence of the Formula:
```
SEQ ID NO:1
ThrGlnAspCysSerPheGlnHisSerProIleSerSerAspPheAlaValLysIleArg
                                                      10 20
GluLeuSerAspTyrLeuLeuGlnAspTyrProValThrValAlaSerAsnLeuGlnAsp
                                                      30 40
GluGluLeuCysGlyGlyLeuTrpArgLeuValLeuAlaGlnArgTrpMetGluArgLeu
                                                      50 60
LysThrValAlaGlySerLysMetGlnGlyLeuLeuGluArgValAsnThrGluIleHis
                                                      70 80
PheValThrLysCysAlaPheGlnProProProSerCysLeuArgPheValGlnThrAsn
                                                      90 100
IleSerArgLeuLeuGlnGluThrSerGluGlnLeuValAlaLeuLysProTrpIleThr
                                                      110 120
ArgGlnAsnPheSerArgCysLeuGluLeuGlnCysGlnProASrSerSerThrLeu
                                                      130
```
wherein the N-terminus is joined to the C-terminus directly or through a linker (L₂) capable of joining the N-terminus to the C-terminus and having new C- and N-termini at amino acids;
28-29
29-30
30-31
31-32
32-33
34-35
36-37
37-38
38-39
39-40
40-41
41-42
42-43
64-65
65-66
66-67
86-87
87-88
88-89
89-90
90-91
91-92
92-93
93-94
94-95
95-96
96-97
97-98
98-99
99-100
100-101
101-102
102-103
respectively;
wherein R₂ is a factor selected from the group consisting of: a colony stimulating factor, a cytokine, a lymphokine, an interleukin, and a hematopoietic growth factor;
wherein L₁ is a linker capable of linking R₁ to R₂; and
said Progenipoietin protein can optionally be immediately preceded by (methionine⁻¹), (alanine⁻¹) or (methionine⁻², alanine⁻¹).

14. A composition comprising the Progenipoietin protein of claim 1, 2, 3, 4, 5, 6, 7, 8, 9, 10, 11, or 12 and at least one pharmaceutically acceptable carrier.

15. A method of treating a patient having a hematopoietic disorder comprising administering to said patient a therapeutically effective amount of the Progenipoietin conjugate of claim 1, 2, 3, 4, 5, 6, 7, 8, 9, 10, 11, or 12.

16. The method of claim 15 wherein said hematopoietic disorder is neutropenia, leukopenia, thrombocytopenia, or anemia.

17. The method of claim 16 wherein said hematopoietic disorder is the result of chemotherapy, radiation therapy, or bone marrow suppressive drugs.

18. A method of treating a patient recovering and/or suffering from a bone marrow transplant, burn, wound, parasite, bacterial or viral infection comprising administering to said patient a therapeutically effective amount of the Progenipoietin conjugate of claim 1, 2, 3, 4, 5, 6, 7, 8, 9, 10, 11, or 12.

19. A method of mobilizing hematopoietic progenitors and stem cells into peripheral blood comprising administering to said patient a therapeutically effective amount of the Progenipoietin conjugate of claim 1, 2, 3, 4, 5, 6, 7, 8, 9, 10, 11, or 12.

20. A method for the production of dendritic cells comprising the steps of;
a) separating hematopoietic progenitor cells or CD34+ cells from other cells; and
b) culturing said hematopoietic progenitor cells or CD34+ cells in a growth medium, comprising the hematopoietic protein of claim 1, 2, 3, 4, 5, 6, 7, 8, 9, 10, 11, or 12.

21. The method of claim 20, further comprising the step of;
c) pulsing said culturing hematopoietic progenitor cells or CD34+ cells with an antigen.

22. The method of claim 20, wherein said growth medium, further comprises; one or more factor selected from the group consisting of; GM-CSF, IL-4, TNF-A, stem cell factor (SCF), flt-3 ligand, IL-3, an IL-3 variant, an IL-3 variant fusion protein, and a multi-functional receptor agonist.

23. The method of claim 21, wherein said growth medium, further comprises; one or more factor selected from the group consisting of; GM-CSF, IL-4, TNF-α, stem cell factor (SCF), flt-3 ligand, IL-3, an IL-3 variant, an IL-3 variant fusion protein, and a multi-functional receptor agonist.

24. A method for treating a human having a tumor, infection or auto-immune disease, comprising the step of; administering the hematopoietic protein of claim 1, 2, 3, 4, 5, 6, 7, 8, 9, 10, 11, or 12 to said human.

25. The method of claim 24, further comprising; administrating one or more factor selected from the group consisting of; GM-CSF, IL-4, TNF-α, stem cell factor (SCF), flt-3 ligand, IL-3, an IL-3 variant, an IL-3 variant fusion protein, and a multi-functional receptor agonist.

26. A method of conjugating a poly(ethylene glycol) to a nucleophile wherein the conjugation is carried out in the presence of an inorganic solvent.
